(19) United States
(12) Patent Application Publication
(10) Pub. No.: US 2009/0142832 A1
(43) Pub. Date: Jun. 4, 2009

(54) Indoles, Derivatives, and Analogs Thereof and Uses Therefor

(76) Inventors: James Dalton, Upper Arlington, OH (US); Duane Miller, Germantown, TN (US); Sunjoo Ahn, Columbus, OH (US); Charles Duke, III, Memphis, TN (US); Dong Jin Hwang, Germantown, TN (US); Jun Yang, Amherst, NY (US)

Correspondence Address: CALFEE HALTER & GRISWOLD, LLP, 800 SUPERIOR AVENUE, SUITE 1400, CLEVELAND, OH, 44114, US

(21) Appl. No.: 11/947,668

(22) Filed: Nov. 29, 2007

(51) Int. Cl. C07D 403/08; C12N 5/00; C07D 209/04

(52) U.S. Cl. 435/375; 548/454; 548/491

(57) ABSTRACT

Indole derivatives and analog compounds and pharmaceutical compositions comprising the same are provided. Also provided are methods of using these compounds to inhibit tubulin polymerization in a cell associated with a proliferative disease or to treat a cancer.

### STATEMENT REGARDING FEDERALLY SPONSORED RESEARCH

This invention was produced in part using funds obtained through Grant DK-065227-02 from the National Institutes of Health. Consequently, the federal government has certain rights in this invention.

### BACKGROUND OF THE INVENTION

Tubulin is an important microtubular protein and an effective molecular target for cancer chemotherapy. Drugs targeting microtubules, including the taxanes and vinca alkaloids, interrupt microtubule spindle-mediated chromosome segregation, arrest the dividing tumor cells in mitosis and subsequently induce apoptosis. The potency, efficacy and widespread clinical use of these agents in a variety of cancers, e.g., breast, ovarian, prostate, lung, leukemias and lymphomas, stand testament to the importance of tubulin and its role in cancer growth. Unfortunately, these drugs also share a common mechanism of drug resistance, namely P-glycoprotein- or ATP binding cassette (ABC) transporter protein-mediated drug efflux, which limits their efficacy in many tumors.

Naturally occurring compounds derived from both food source and non-food source plants have been tested and often have demonstrated an anticancer effect against various cancers. Derivatives and analogs of these plant compounds are constantly being isolated or synthesized to find more efficacious anticancer agents. Recently, the compound indole-3-carbinol, a phytonutrient derived from cruciferous vegetables, such as broccoli, brussel sprouts or cabbage, has been studied as a potential anticancer therapeutic against breast, cervical, prostate, and colon cancers.

Other indole derivatives have been synthesized. U.S. Pat. No. 6,638,964 discloses indole derivatized with substituted sulfonamides useful to treat malignancies and autoimmune diseases. U.S. Pat. No. 6,812,243 discloses highly substituted bisindoles useful as tyrosine kinase inhibitors to treat cell proliferative diseases.

However, naturally occurring or synthetic indole compounds used as anticancer agents may have drawbracks due to large dosages, loss of anticancer activity from metabolic breakdown, or toxicity. Attempts to develop effective indole derivatives that can be easily administered in reasonable doses, that retain the ability to inhibit activities associated with onset of a cell proliferative disease, and that have improved stability, increased clinical effectiveness, consistent results, and minimal toxicity and side effects are continuously ongoing.

Thus, the prior art is still deficient in the lack of indole derivatives and analogs useful as therapeutics.

### SUMMARY OF THE INVENTION

In accordance with embodiments of the present invention, compounds are provided. The compounds have a structural formula of

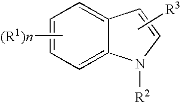

where:

R¹ is H, halide, CF₃, NO₂, OH, —OCH₃, or CN alkyl, alkenyl, O-alkyl, and O-aryl, and n is 0, 1, 2, 3, or 4;

R² is H or —SO₂Ph;

R³ is phenyl substituted at C3 or C5 with R⁴; R⁸R⁹; R¹²R¹³; 2-, 3- or 6-indolyl substituted at C1, C2, or C3 with 2-, 3- or 6-indolyl, either of the indolyl moiety independently substituted at C1 with R², at C4, C5, or C6 with R¹ or with a combination thereof; or naphthyl substituted at C5, C6, or C7 with 2-, 3- or 6-indolyl or unsubstituted, the indolyl moiety independently substituted at C1 with R², at C4, C5, or C6 with R¹ or with a combination thereof;

R⁴ is R⁵; C₁₋₃alkylene-R⁵; C(O)R⁶; CH═CH—C(R⁷)—R⁶; —C(O)—R⁷—R⁶; —O—C(R⁷)—R⁶; R^{8;} R⁷R⁸-(2-, 3-, or 6-indolyl); R⁸-(2-, 3- or 6-indolyl), the indolyl moiety independently substituted at C1 with R², at C4, C5 or C6 with R¹ or with a combination thereof; R⁸R⁹or R¹²R¹³;

R⁵ is OH, NO₂, NH₂, —NH—C₁₋₃alkyl, N═N═N, CN, or OR⁶;

R⁶ is H, C₁₋₃alkyl, or a 5- or 6-membered ring independently substituted at C2, C3, C4, C5, or C6 with R1;

R⁷ is O, S or NH;

R⁸ is —CH₂, —CH₂OH, C═O, C═S, C═CH₂, C═NOH, C═N(NH₂);

R⁹ is phenyl independently substituted at C3 with R¹⁰ and at C4 and C5 with R¹¹; thiazolyl substituted at C4 with —C(O)OCH₃ or naphthyl substituted at C5, C6, or C7 with 2-, 3- or 6-indolyl or unsubstituted, the indolyl moiety independently substituted at C1 with R², at C4, C5, or C6 with R¹ or with a combination thereof;

R¹⁰ is H, OH, —OCH₃, phenyl, naphthyl or forms a dioxolyl ring with R¹¹ at C4;

R¹¹ is H, OH, or —OCH₃;

R¹² is pyrrolyl, furanyl, thienyl, or cyclopentadienyl;

R¹³ is —C(O)-2-, 3-, or 6-indolyl, —C(O)-imidazole, —C(O)-thiazole, —C(O)-oxazole, —C(O)-isoxazole, —C(O)-benzoxazole, —C(O)-pyrrole, —C(O)-furan, —C(O)-oxazoline, —C(O)-oxazolidine, —C(O)-oxadiazole, C(O)-napthyl or —C(O)phenyl, each independently substituted with at C2, C3, C4, C5, or C6 with R¹.

These compounds may also be in the form of a pharmacologically acceptable salt or hydrate. These compounds may be formulated as a pharmaceutical composition with a pharmaceutically acceptable carrier.

In accordance with yet further embodiments, methods of inhibiting tubulin polymerization in a cell associated with a cell proliferative disease in a subject are also provided. The methods can comprise contacting the cell associated with the cell proliferative disease with a pharmacologically effective amount of the compound or of the pharmaceutical compositions thereof described herein.

In yet further embodiments, methods of treating a cancer in a subject are provided. The methods can comprise administering a pharmacologically effective amount of the compounds or of the pharmaceutical compositions thereof described herein to the subject where the compounds inhibit growth of cancer cells thereby treating the cancer.

### BRIEF DESCRIPTION OF THE SEVERAL VIEWS OF THE DRAWINGS

The following detailed description of embodiments of the present invention can be best understood when read in conjunction with the following drawings, where like structure is indicated with like reference numerals and in which:

FIGS. 1A-1J depict representative synthetic schemes and representative structures for compounds according to the present invention. Synthetic schemes are shown for compounds 10, 11 and 13 in FIG. 1A. Structures for compounds 14-31 are shown in FIG. 1B. Synthetic schemes for preparing compounds of at least one of the structures 14-31 are shown in FIGS. 1C-1J.

FIGS. 2A-2C illustrate that compound 13 induces apoptosis (FIG. 2A), decreases anti-apoptosis proteins (FIG. 2B) and induces DNA fragmentation (FIG. 2C) in LnCap and PC-3 cells.

FIGS. 3A-3B illustrate that compound 13 induces G2/M phase arrest (FIG. 3A) in LNCaP cells and inhibits polymerization of tubulin proteins in vitro (FIG. 3B).

FIG. 4 illustrates the effect of 50, 100 and 200 mg/kg of compound 13 on body weight of ICR mice.

FIG. 5 illustrates the mean plasma concentration-time profile of compound 13 in mice.

FIG. 6 illustrates the antitumor activity of compound 13 against a PC-3 xenograft in Balb/c mice.

### DETAILED DESCRIPTION OF EMBODIMENTS

The present invention will now be described with occasional reference to the specific embodiments of the invention. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

As used herein, the term “alkyl” shall refer to optionally substituted straight, branched, cyclic, saturated, or unsaturated hydrocarbon chains.

As used herein, the term “halogen” or “halide” shall refer to fluorine, chlorine, bromine, or iodine.

As used herein, the term “aryl” shall refer to optionally substituted aromatic mono- or bicyclic hydrocarbons. Heteroaryl shall refer to an aryl compound with one or more heteroatoms, e.g., nitrogen, sulfur or oxygen, in the aromatic ring structure.

As used herein, the term “contacting” refers to any suitable method of bringing an inhibitory agent into contact with a cell. In some examples the cell is an abnormally proliferating cell. In vitro or ex vivo this is achieved by exposing the cells to the inhibitory agent in a suitable medium. For in vivo applications, any known method of administration is suitable.

As used herein, the term “treating” or the phrase “treating a cancer” includes, but is not limited to, halting the growth of cancer cells, killing the cancer cells or a mass comprising the same, or reducing the number of cancer cells or the size of a mass comprising the same. Halting the growth refers to halting any increase in the size or the number of cancer cells or in a mass comprising the same or to halting the division of the cancer cells. Reducing the size refers to reducing the size of a mass comprising the cancer cells or the number of or size of the same cells. As would be apparent to one of ordinary skill in the art, the term “cancer” or “cancer cells” or “tumor” refers to examples of neoplastic cell proliferative diseases and refers to a mass of malignant neoplastic cells or a malignant tissue comprising the same.

As used herein, the term “inhibiting” or “inhibition” of tubulin polymerization in cells associated with a cell proliferative disease, e.g., cells comprising a cancer or tumor or malignant or abnormally proliferating cells, shall include partial or total inhibition of tubulin formation and also is meant to include decreases in the rate of proliferation or growth of the cells associated with the cell proliferative disease. The biologically inhibitory dose of the composition of the present invention may be determined by assessing the effects of the test element on tubulin polymerization in target malignant or abnormally proliferating cells in tissue culture or cell culture, on tumor growth in animals or any other method known to those of ordinary skill in the art.

As used herein, the term “subject” refers to any target of the treatment.

In accordance with embodiments of the present invention, indole derivative compounds are provided. The compounds have the structural formula:

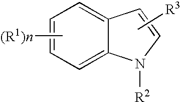

where:

R¹ is H, halide, CF₃, NO₂, OH, —OCH₃, or CN alkyl, alkenyl, O-alkyl, and O-aryl, and n is 0, 1, 2, 3, or 4;

R² is H or —SO₂Ph;

R³ is phenyl substituted at C3 or C5 with R⁴; R⁸R⁹; R¹²R¹³; 2-, 3- or 6-indolyl substituted at C1, C2, or C3 with 2-, 3- or 6-indolyl, either of the indolyl moiety independently substituted at C1 with R², at C4, C5, or C6 with R¹ or with a combination thereof; or naphthyl substituted at C5, C6, or C7 with 2-, 3- or 6-indolyl or unsubstituted, the indolyl moiety independently substituted at C1 with R², at C4, C5, or C6 with R¹ or with a combination thereof;

R⁴ is R⁵; C₁₋₃alkylene-R⁵; C(O)R⁶; CH═CH—C(R⁷)—R⁶; —C(O)—R⁷—R⁶; —O—C(R⁷)—R⁶; R^{8;} R⁷R⁸-(2-, 3-, or 6-indolyl); R⁸-(2-, 3- or 6-indolyl), the indolyl moiety independently substituted at C1 with R², at C4, C5 or C6 with R¹ or with a combination thereof; R⁸R⁹or R¹²R¹³;

R⁵ is OH, NO₂, NH₂, —NH—C₁₋₃alkyl, N═N═N, CN, or OR⁶;

R⁶ is H, C₁₋₃alkyl, or a 5- or 6-membered ring independently substituted at C2, C3, C4, C5, or C6 with R1;

R⁷ is O, S or NH;

R⁸ is —CH₂, —CH₂OH, C═O, C═S, C═CH₂, C═NOH, C═N(NH₂);

R⁹ is phenyl independently substituted at C3 with R¹⁰ and at C4 and C5 with R¹¹; thiazolyl substituted at C4 with —C(O)OCH₃ or naphthyl substituted at C5, C6, or C7 with 2-, 3- or 6-indolyl or unsubstituted, the indolyl moiety independently substituted at C1 with R², at C4, C5, or C6 with R¹ or with a combination thereof;

R¹⁰ is H, OH, —OCH₃, phenyl, naphthyl or forms a dioxolyl ring with R¹¹ at C4;

R¹¹ is H, OH, or —OCH₃;

R¹² is pyrrolyl, furanyl, thienyl, or cyclopentadienyl;

R¹³ is —C(O)-2-, 3-, or 6-indolyl, —C(O)-imidazole, —C(O)-thiazole, —C(O)-oxazole, —C(O)-isoxazole, —C(O)-benzoxazole, —C(O)-pyrrole, —C(O)-furan, —C(O)-oxazoline, —C(O)-oxazolidine, —C(O)-oxadiazole, C(O)-napthyl or —C(O)phenyl, each independently substituted with at C2, C3, C4, C5, or C6 with R¹; or a pharmacologically acceptable salt or hydrate thereof.

In some examples R¹ may be H, R³ may be phenyl substituted at C3 or C5 with R⁴, and R⁴ may be R⁸. Examples include, but are not limited to, compounds having a structure of:

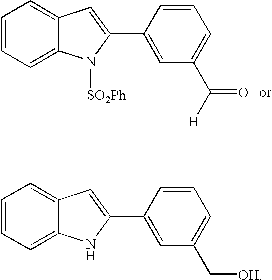

In other examples, R¹ may be H or F and R³ may be phenyl substituted at C3 or C5 with R⁴, and R⁴ may be —R⁸-(2- or 3-indolyl). Examples include, but are not limited to, compounds having a structure of:

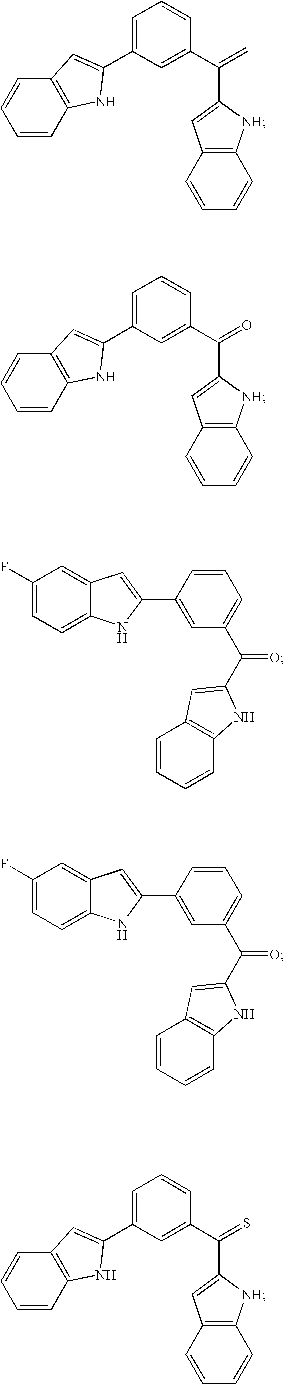

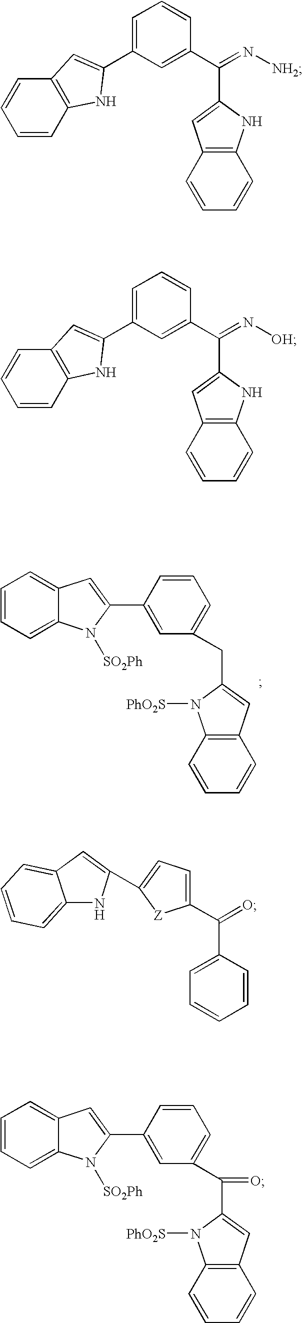

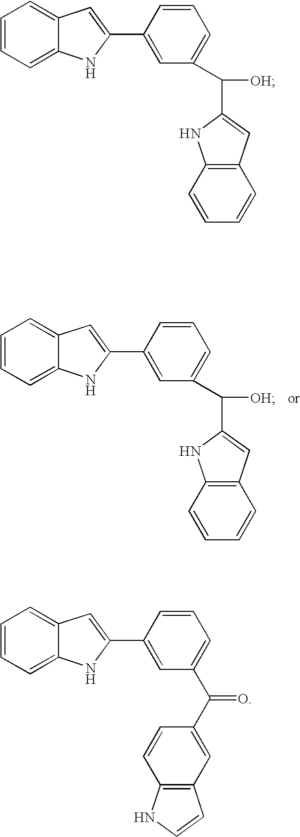

In yet other examples, R³ may be phenyl substituted at C3 or C5 with R⁴ and R⁴ may be R⁷R⁸-(2-, 3-, or 6-indolyl). Examples of suitable compounds include, but are not limited to, those having the structure:

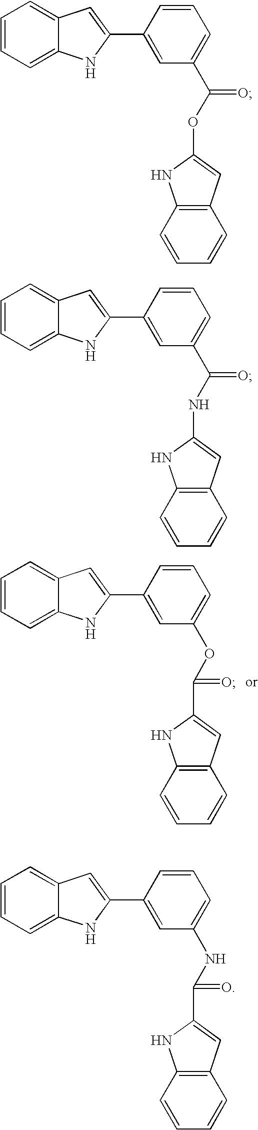

In yet further examples, R³ may be phenyl substituted at C3 or C5 with R⁴ and R⁴ may be R⁸R⁹. Examples of suitable compounds include, but are not limited to, those having the structure:

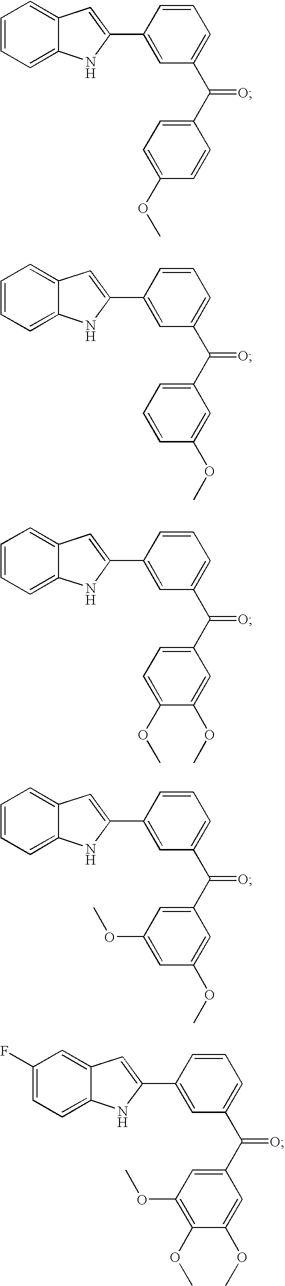

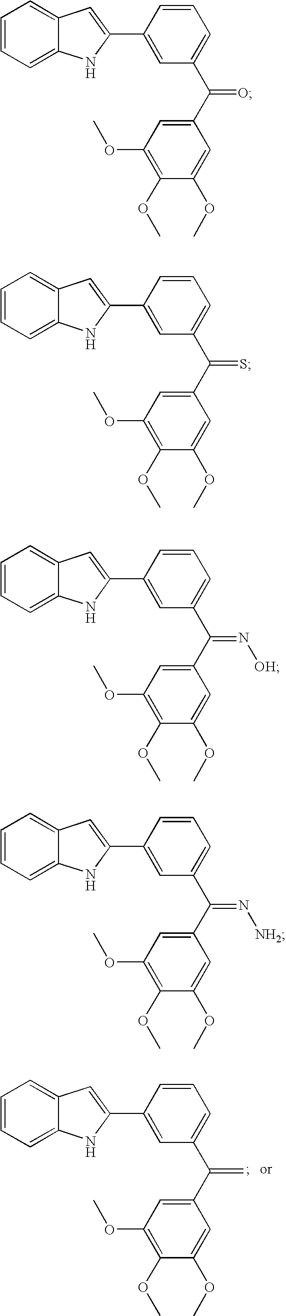

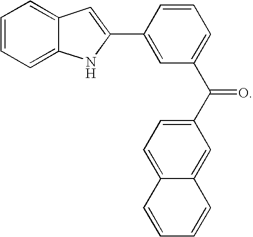

In some examples, the compound may have a structure of

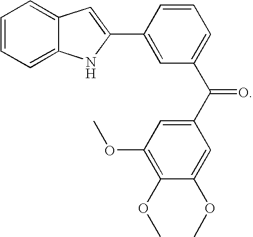

In other examples, R³ may be 2-, 3- or 6-indolyl. Examples of suitable compounds include, but are not limited to, those having a structure of:

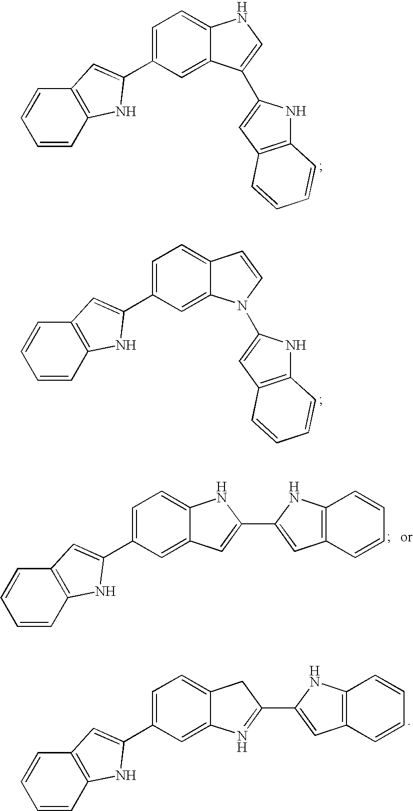

In yet other examples, R³ is napthyl. Examples of suitable compounds include, but are not limited to, those having a structure of:

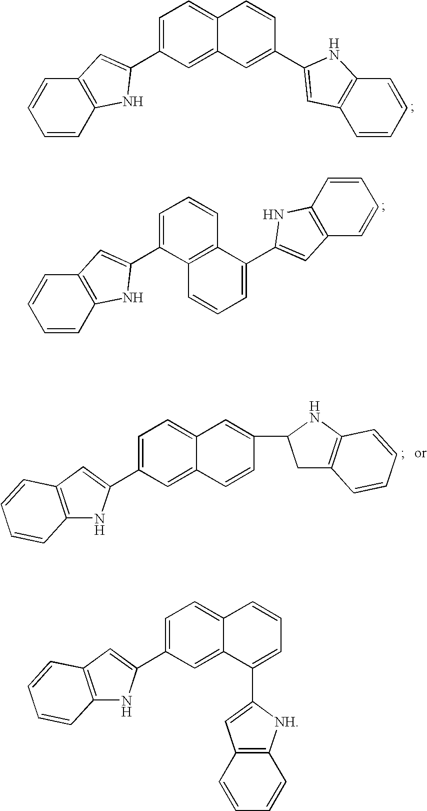

In yet other examples, R³ is R⁸R⁹. Examples of suitable compounds include, but are not limited to, those having a structure of:

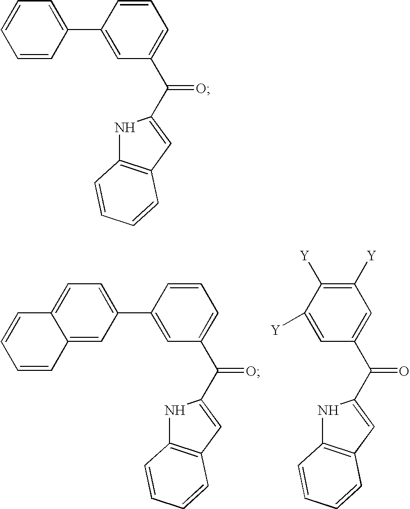

wherein Y is independently selected from H, OH, OCH₃; or

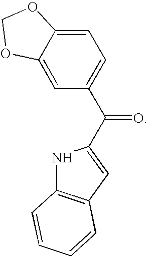

In yet other examples, R³ is R¹²R¹³. Examples of suitable compounds include, but are not limited to, those having a structure of:

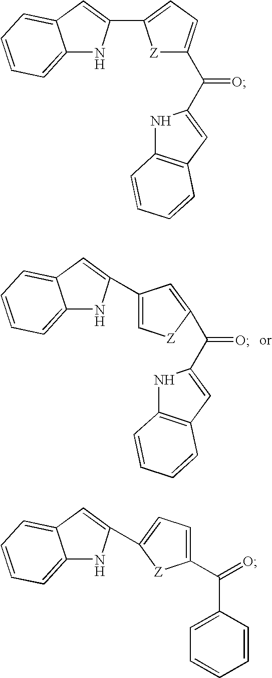

and wherein Z is independently selected from S, O, NH, and CH₂.

These compounds may be synthesized in any suitable manner. For example, the compounds may be synthesized using the techniques as described in the Examples presented herein. Numbering of the carbon atoms uses standard protocol where the nitrogen heteroatom in indole is C1 and the carbon atom in the phenyl moiety linked to C2 in indole is C1. This numbering protocol also is used with any substituent ring structure comprising these indole or diindole derivatives or analogs, such as, a cyclic alkyl, an aryl or a heteroaryl moiety.

In some embodiments the compound or a combination of compounds, with a pharmaceutically acceptable carrier, may comprise a pharmaceutical composition.

In other embodiments, there is provided methods of inhibiting tubulin polymerization in a cell associated with a cell proliferative disease comprising contacting the cell associated with the cell proliferative disease with a pharmacologically effective amount of at least one compound described herein. In this embodiment, the cell proliferative disease may be a cancer. Representative examples of cancers include prostate cancer, colon cancer or breast cancer.

In still other embodiments, there is provided methods of treating a cancer in a subject comprising administering a pharmacologically effective amount of at least one compound as described herein to the subject, where the compound inhibits growth of cancer cells thereby treating the cancer. In this embodiment, representative examples of a cancer include prostate cancer, colon cancer or breast cancer.

The compounds provided herein may be useful as therapeutics to inhibit growth of abnormally proliferating cells in a cell proliferative disease by inhibiting tubulin or tubulin polymerization in the cell while circumventing ATP binding cassette transporter mediated multi-drug resistance. It is contemplated that contacting the abnormally proliferating cells with this compound is effective to induce apoptosis and/or cell cycle arrest. Thus, the compounds of the present invention may be useful in treating cancers in a subject. In some examples, the subject is a mammal. In other examples, the subject is a human. Examples of cancers may include, but are not limited to, prostate cancer, colon cancer or breast cancer.

Dosage formulations of these compounds or a pharmacologically acceptable salt or hydrate thereof may comprise conventional non-toxic, physiologically or pharmaceutically acceptable carriers or vehicles suitable for the method of administration. These compounds or pharmaceutical compositions thereof may be administered independently one or more times to achieve, maintain or improve upon a pharmacologic or therapeutic effect derived from these compounds or other anticancer drugs or agents. It is well within the skill of an artisan to determine dosage or whether a suitable dosage comprises a single administered dose or multiple administered doses. An appropriate dosage depends on the subject's health, the progression or remission of the cancer, the route of administration and the formulation used.

The following examples are given for the purpose of illustrating various embodiments of the invention and are not meant to limit the present invention in any fashion.

### EXAMPLE 1

### Synthesis of Compounds

General Synthetic Scheme

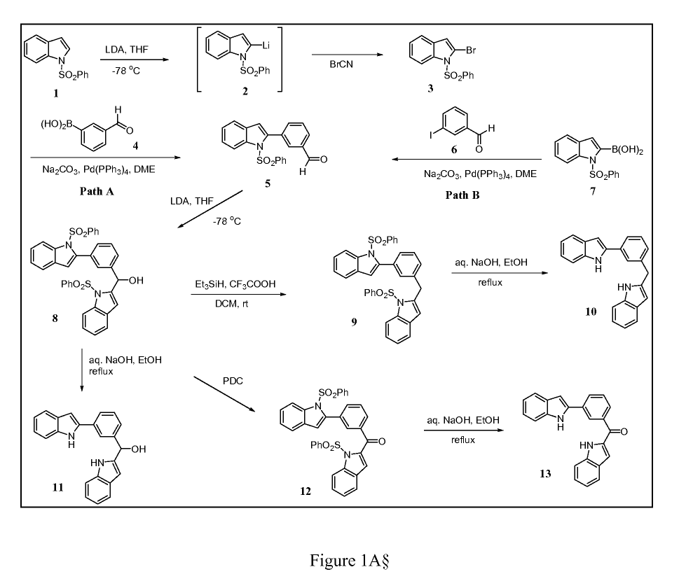

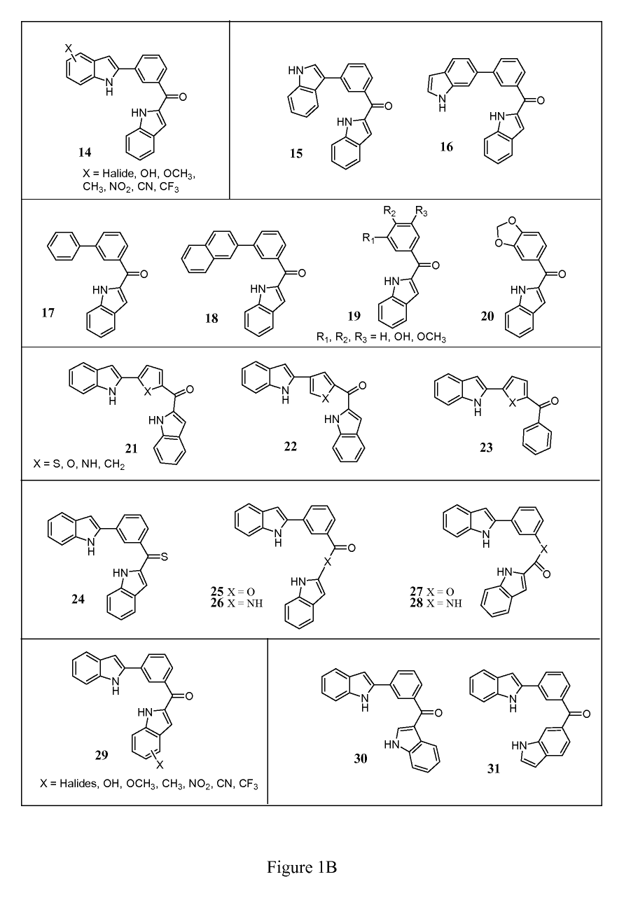

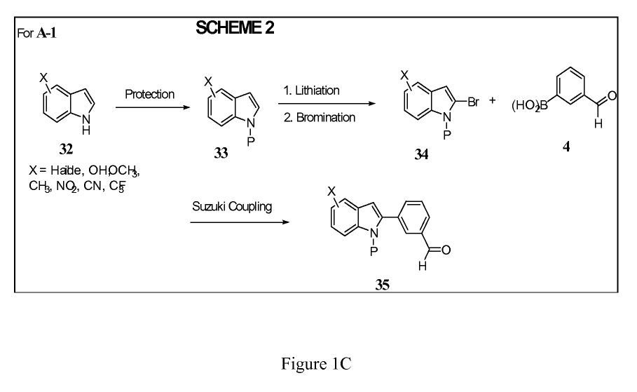

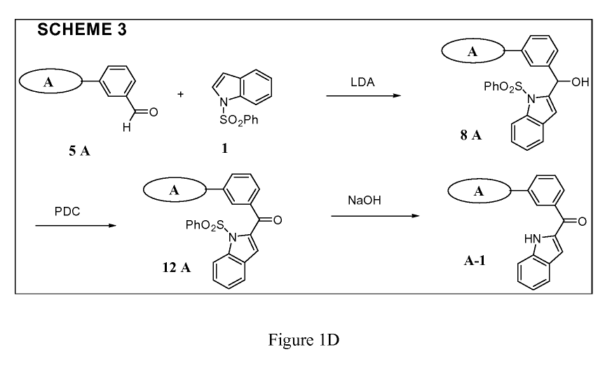

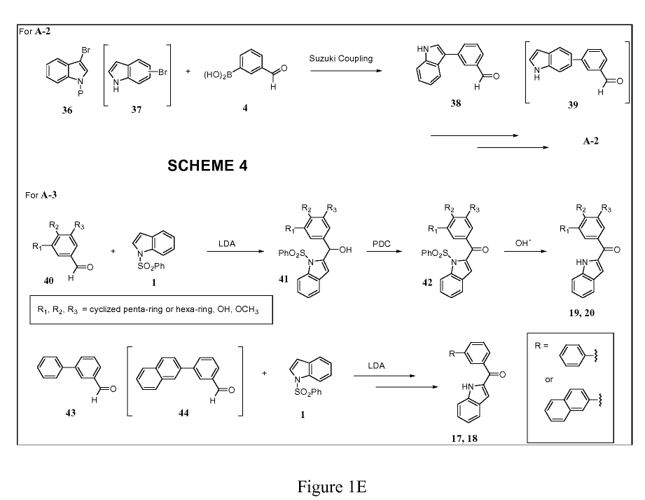

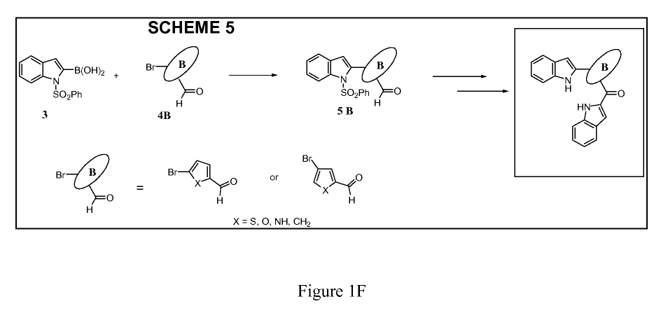

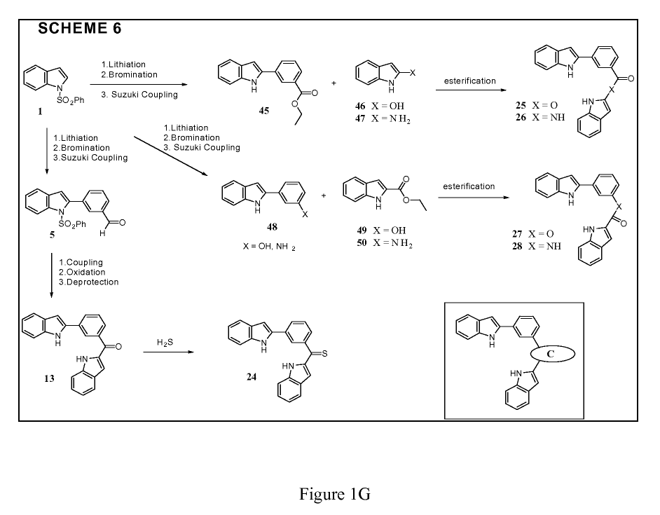

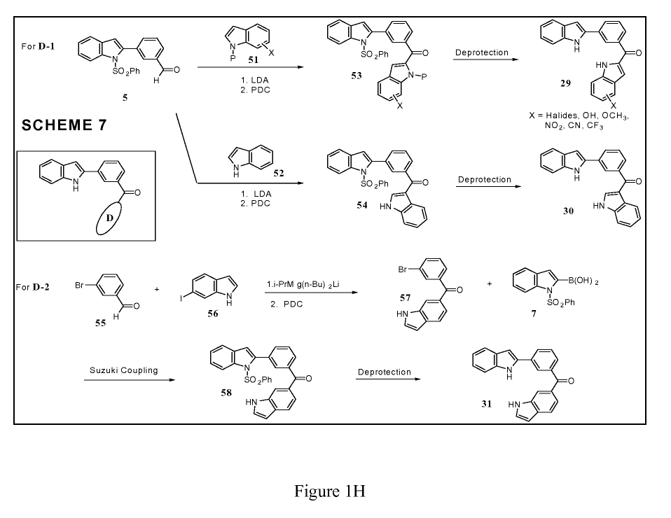

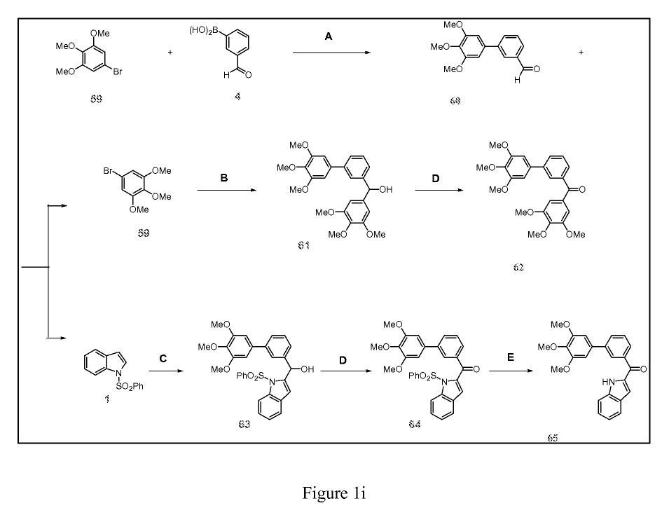

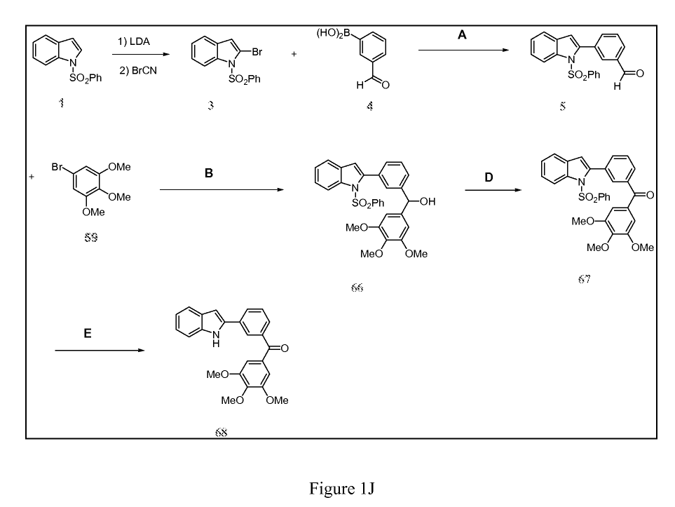

Known synthetic methods are used to synthesize the compounds 10, 11, and 13 as shown in FIG. 1. As shown in the synthetic scheme in FIG. 1, target diindoles 10, 11 and 13 bridged via methylphenyl linkers were prepared by removing protecting group bezenesulfonyl under reflux of ethanolic NaOH solution from corresponding precursor compounds 9, 8 and 12 using a general procedure described below. Intermediate compound 8 is key to the subsequent synthesis of compounds 10, 11 and 13. Compound 8 is synthesized from the coupling of protected indole 1 with protected indole benzaldehyde compound 5 in the presence of lithium diisopropyl amide (LDA) as a 94% yield. Compound 5 may be synthesized using two different Suzuki coupling pathways, path A and path B.

For path A, the lithiation of protected indole 1 by LDA to yield indole 2 followed by bromination with cyanogen bromide (BrCN) produced bromoindole 3. The synthesized bromoindole 3 was coupled with aldehydophenylboric acid 4 to yield compound 5. For path B, compound 5 was prepared using commercially available iodophenylaldehyde 6 and protected indole boric acid 7.

Using triethylsilane and trifluoroacetic acid (TFA), the phenylmethanol linker in compound 8 was additively reduced to phenylmethylene in compound 9 at room temperature as a 67% yield. In this reduction triphenylsilane, as another silylating agent was poor yield because of resistance for its bulky group. Methylphenyl-linked diindole compound 10 was afforded from protected diindole 9 by the general procedure. By treating compound 8 with sodium hydroxide (10 eq.) under reflux ethanol for 20 hr, the free methanol-linked diindole 11 was produced.

By oxidizing the phenylmethanol linker in compound 8 with pyridinium dichromate (PDC) in dimethylformamide (DMF), the protected phenylmethanone linked compound 12 was synthesized as a 73% yield. Phenylmethanone-linked compound 13 was synthesized by the general procedure from compound 12 as an 83% yield.

### Synthesis of 2-bromo-1-(benzenesulfonyl)indole (3)

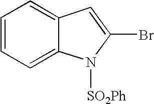

Compound 12 was prepared by Ketcha's method (1). Calculated mass 334.96, [M−H] 334.1. Anal. calc. for C₁₄H₁₀BrNO₂S; C, H, N.

### Synthesis of 3-(1-(benzenesulfonyl-1H-indol-2-yl)benzaldehyde (5)

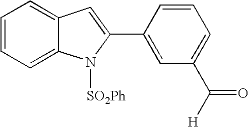

Compound 5 was produced by Suzuki's coupling using path A or path B. Path A and path B utilize the same procedure of coupling an organoboronic acid with an aryl halide, but path A uses the aryl halide compound 3 and the organoboronic acid compound 4 as described herein. Path B uses the aryl halide 1-iodo-3-formyl benzene 6 and the organoboronic acid 1-(phenylsulfonyl)-1H-indol-2-yl-boronic acid 7. Compound structures are shown in FIG. 1.

A mixture of 2-bromo-1-(phenylsulfonyl)-1H-indole 3 (330 mg, 0.99 mmol), tetrakis)triphenylphosphine)palldium(0) (34 mg, 0.3 μmol) and 3-formylphenyl boric acid 4 (177 mg, 1.18 mmol) in dimethoxyethan (DME) (10 ml) with sodium carbonate (1 ml of 2 M in deoxygenated water) was stirred and heated to reflux for 2 hr until bromoinodole 3 was not detected on TLC. The mixture was cooled to room temperature and poured into EtOAc (20 ml) and extracted with EtOAc. The combined organic layers were washed with saturated NH4Cl and water and dried over MgSO₄. The solvent was removed in vacuo and then purified by flash column chromatography on silica gel using EtOAc/Hx (1:5) as an eluent to give compound 5 (336 mg, 94%) as a yellowish solid. Mp 126-138° C.; Anal. calc. for C₂₁H₁₅NO₃S; C, H, N. ¹H NMR (CDCl₃) δ 10.1 (bs, 1H, CHO), 8.33 (d, J=8.1 Hz, 1H, ArH), 7.98 (s, 2H, ArH), 7.85 (d, J=7.2 Hz, 1H, ArH), 7.63 (t, 1H, J=7.8 Hz, ArH), 7.51-7.29 (m, 8H, ArH) 6.66 (s, 1H, ArH), ¹³C NMR (CDCl₃) δ 192.1, 140.5, 138.6, 137.6, 136.6, 136.1, 134.0, 133.7, 131.1, 130.6, 130.0, 129.0 (2C), 128.5, 126.8 (2C), 125.6, 1254.9, 121.2, 116.8, 114.9.

### Synthesis of (1benzenesulfonyl-1H-indol-2-yl)-[3-(1-benzenesulfonyl-1H-indol-2-yl)phenyl]methanol (8)

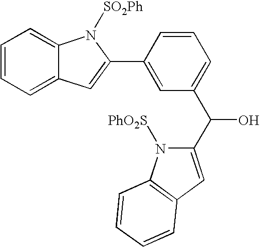

To a solution of protected indole 1 (2.37 g, 6.56 mmol) in 30 ml tetrahydrofuran (THF), 2.0 M LDA solution (4.75 ml, 9.5 mmol) in THF was added within 10 min at −78° C. The solution was stirred at 0° C. for 30 min and subsequently cooled to −78° C. At this temperature, aldehydoindole 5 (2.03 g, 7.88 mmol) dissolved in dry THF (10 ml) was added. The resulting mixture was stirred overnight and allowed to warm to room temperature. The solution was poured into 100 ml of EtOAc. The combined organic layers were washed with saturated NH4Cl and water and dried over MgSO4. The solvent was removed in vacuo and then purified by flash column chromatography on silica gel using EtOAc/Hx (1:3) as an eluent to give compound 8 (3.32 g, 82%) as a yellowish solid. Calculated Mass 618.13, [M+Na⁺] 641.2; Mp 81-83° C.; Anal. calc. for C₃₅H₂₆N₂O₅S₂; C, H, N; ¹H NMR (CDCl₃) δ 8.29 (d, J=8.4 Hz, 1H, ArH), 8.09 (d, J=8.4 Hz, 1H, ArH), 7.73 (d, J=7.5 Hz, 2H, ArH), 7.61 (s, 1H, ArH), 7.56-7.06 (m, 17H, ArH), 6.57 (s, 1H, ArH), 6.48 (s, 1H, ArH), 6.42 (s, 1H, CH), 3.64 (bs, 1H, OH); ¹³C NMR (CDCl₃) δ 143.3, 141.3, 140.0, 138.0, 137.8, 136.9, 136.8, 133.5, 133.1, 132.1, 130.1, 129.6, 128.9, 128.6 (2C), 128.5, 128.1 (2C), 127.1, 126.9, 126.2 (2C), 125.9 (2C), 124.7, 124.5, 124.1, 123.5, 121.0, 120.4, 116.1, 114.2, 113.7, 112.0, 68.8.

### Synthesis of (1-benzenesulfonyl-1H-indol-2-yl)-[3-(1-Benzenesulfonyl-1H-indol-2-yl)phenyl]methane (9)

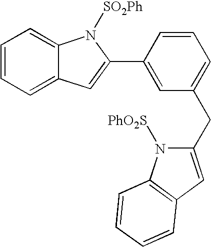

After stirring a solution of compound 8 (201 mg, 0.32 mmol) and triethylsilane (0.1 ml, 0.65 mmol) in 5 ml dry CH₂Cl₂ for 30 min, TFA (0.16 ml, 1.95 mmol) was added. The solution was stirred for 1 h at room temperature, 10 ml H₂O was added to the solution and the solution was carefully neutralized with solid Na₂CO₃ with ice cooling. The organic phase was separated, dried over Na₂SO₄, and concentrated and then purified by flash column chromatography on silica gel using EtOAc/Hx (1:5) as an eluent to give compound 9 (130 mg, 67%) as a yellowish solid. Calculated Mass 602.13, [M+Na⁺] 625.2; Mp 76-78° C.; Anal. calc. for C₃₅H₂₆N₂O₅S₂ 0.2 C₄H₈O₂; C, H, N; ¹H NMR (CDCl₃) δ 8.30 (d, J=8.1 Hz, 1H, ArH), 8.16 (d, J=8.1 Hz, 1H, ArH), 7.67 (d, J=7.8 Hz, 2H, ArH), 7.51-7.12 (m, 18H, ArH), 6.52 (s, 1H, ArH), 6.30 (s, 1H, CH), 4.34 (s, 2H, CH₂); ¹³C NMR (CDCl₃) δ 141.4, 140.1, 138.5, 137.8, 136.9, 136.7, 134.5, 133.2, 133.0, 132.2, 130.6, 130.2, 129.1, 129.0, 128.7 (2C), 128.5 (2C), 128.0, 127.1, 126.2(2C), 125.9 (2C), 124.4, 123.9, 123.7, 123.1, 120.2, 120.0, 116.1, 114.2, 113.4, 110.9, 34.6.

General Procedure for Preparation of Compounds 10, 11 and 13

To a solution of compound protected indole (0.56 mmol) in 10 ml ethanol was added a 10% solution of NaOH (227 mg, 5.68 mmol) and the mixture was refluxed for 20 h. Then, ethanol was evaporated, brine and CH₂Cl₂ were added, the organic phase was extracted with CH₂Cl₂ and then was purified by flash column chromatography on silica gel using EtOAc/Hx (1:1) or CH₂Cl₂/Hx (1:1) as an eluent to give target free indole compound (69%˜91%).

### Synthesis of (1H-Indol-2-yl)-[3-(1H-indol-2-yl)phenyl]methane (10)

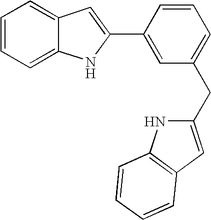

Compound 10 is synthesized from compound 9 by the general procedure described above. Brown solid; Yield 91%; Calculated Mass 322.15, [M−H] 321.2; Mp 193-194° C.; Anal. calc. for C₃₅H₂₆N₂O₅S₂; C, H, N; ¹H NMR (CDCl₃) d 8.30 (bs, 1H, NH), 7.82 (bs, 1H, NH), 7.63-7.54 (m, 4H, ArH), 7.42-7.37 (m, 2H, ArH), 7.27-7.01 (m, 6H, ArH), 8.82 (s, 1H, ArH), 6.34 (s, 1H, ArH), 4.19 (s, 2H, CH₂); ¹³C NMR (CDCl₃) δ 138.8, 136.9, 136.8, 136.3, 135.8, 132.4, 128.9, 128.7, 128.1, 127.7, 124.8, 123.2, 121.9, 120.9, 120.1, 119.9, 119.5, 119.3, 110.4, 110.0, 100.9, 99.7, 34.3.

### Synthesis of (1H-indol-2-yl)-[3-(1H-indol-2-yl)phenyl]methanol (11)

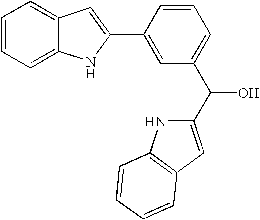

Compound 11 is synthesized from compound 8 by the general procedure described above. Yield 69%; Brown solid; Calculated Mass 338.40, [M−H] 337.2; Anal. calc. for C₂₃H₁₈N₂O; C, H, N; Mp 82-85° C.; ¹H NMR (CDCl₃) δ 8.37 (bs, 1H, NH), 8.26 (bs, 1H, NH), 7.71 (s, 1H, ArH), 7.60-7.06 (m, 11H, ArH), 6.80 (s, 1H, CH), 6.32 (s, 1H, ArH), 5.97 (s, 1H, ArH). ¹³C NMR (CDCl₃) d 141.8, 139.3, 136.9, 136.4, 132.3, 128.8, 128.6, 128.1, 127.5, 125.4, 124.5, 122.3, 122.0, 121.8, 120.2 (2C), 119.8, 119.5, 110.6, 110.5, 100.7, 99.8, 70.2.

### Synthesis of (1-benzenesulfonyl-1H-indol-2-yl)-[3-(1-Benzenesulfonyl-1H-indol-2-yl)phenyl]methanone (12)

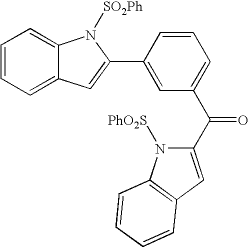

To a solution of compound 8 (325 mg, 0.53 mmol) in dry DMF (10 ml) pyridinium dichromate (PDC, 1.28 mg, 3.4 mmol) was added at 0° C. The mixture was stirred for 20 h at room temperature. H₂O and CH₂Cl₂ were added, the layers were separated, and the aqueous phase was extracted with CH₂Cl₂. The combined organic extracts were washed with water and dried over MgSO₄. The solvent was evaporated and then purified by flash column chromatography on silica gel using EtOAc/Hx (1:3) as an eluent to give compound 12 (225 mg, 70%) as a yellowish solid. Calculated mass 616.11, [M+Na⁺] 639.2; Mp 189-190° C.; Anal. calc. for C₃₅H₂₄N₂O₅S₂ 0.2 C₄H₈O₂; C, H, N; ¹H NMR (CDCl₃) δ 8.33 (d, J=8.4 Hz, 1H, ArH), 8.20-8.06 (m, 4H, ArH), 7.85 (d, J=8.4 Hz, 1H, ArH), 7.84-7.27 (m, 16H, ArH), 7.13 (s, 1H, ArH), 6.66 (s, 1H, ArH); ¹³C NMR (CDCl₃) δ 186.3, 140.1, 137.9, 137.8, 137.4, 137.2, 136.8, 136.4, 135.1, 133.4, 133.2, 132.2, 130.9, 129.9, 129.6, 128.5 (2C), 128.3 (2C), 128.1, 127.3, 127.0 (2C), 126.7, 126.1 (2C), 124.7, 124.0, 123.8, 122.2, 120.4, 116.8, 116.1, 114.6, 114.0.

### Synthesis of (1H-indol-2-yl)-[3-(1H-indol-2-yl)phenyl]methanone (13)

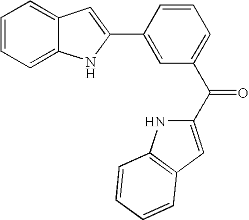

Compound 13 is synthesized from compound 12 by the general procedure described above. Yield 83%; Brown solid; Calculated Mass 336.39, [M−H] 335.3; Mp 206-207° C.; Anal. calc. for C₂₃H₁₆N₂O.0.2 C₄H₈O₂; C, H, N; ¹H NMR (DMSO) d 8.38 (bs, 1H, NH), 8.18 (bs, 1H, NH), 7.86-7.04 (m, 13H, ArH), 5.77 (s, 1H, ArH). ¹³C NMR (DMSO) δ 186.0, 138.8, 138.1, 137.3, 136.6, 134.2, 129.1, 128.6, 128.4, 127.7, 127.0, 125.8, 124.8, 123.0, 121.9, 120.3, 120.2, 119.5, 112.7, 112.4, 111.4, 99.6.

General Procedure A for Preparation of Compounds 60 and 5 (FIG. 1i and 1J)

A mixture of arylbromide 1 or compound 3 (0.99 mmol), tetrakis(triphenylphosphine)palladium (0) (34 mg, 0.3 μmol), and 3-formylphenyl boric acid 4 (177 mg, 1.18 mmol) in DME (10 mL) with sodium carbonate (1 mL of 2 M in deoxygenated water) was stirred and heated to reflux for 2 hr until arylbromide 1 or compound 3 was not detectable on TLC. The mixture was cooled to room temperature and poured into EtOAc (20 mL), extracted with EtOAc. The combined organic layers were washed with sat. NH₄Cl, water and dried over anhydrous MgSO₄. The solvent was removed under reduced pressure and then purified by flash column chromatography on silica gel using EtOAc/Hexane (1/5, v/v) as an eluent to give target aldehyde compounds.

General Procedure B for Preparation of Compounds 61 and 66 (FIG. 1i and 1J)

To a solution of bromide 59 (1.38 mmol) in dry THF (10 mL) cooled to −78° C. was added n-BuLi (0.61 mL, 2.5 M, 1.1 eqiv) under argon atmosphere. The solution was stirred for 30 min, aldehyde 60 (1.38 mmol) in anhydrous THF was added, and the solution stirred for 16 h. Water was added to quench the reaction. The reaction solution was extracted with EtOAc, dried with anhydrous MgSO₄. The solvent was removed under reduced pressure and then purified by flash column chromatography on silica gel using EtOAc/Hexane (1/1, v/v) as an eluent to give target compounds.

General Procedure D for Preparation of Compounds 62, 64 and 67 (FIGS. 1i and 1J)

To the solution of compound 61, 63, 66 (0.53 mmol) in dry DMF (10 mL) was added pyridinium dichromate (PDC, 1.28 mg, 3.4 mmol) at 0° C. The mixture was stirred for 20 h at room temperature. Then, H₂O and CH₂Cl₂ were added, the layers were separated, and the aqueous phase was extracted with CH₂Cl₂. The combined organic extracts were washed with water and dried over anhydrous MgSO₄, and the solvent was evaporated, and then purified by flash column chromatography on silica gel using EtOAc/Hexane (1/3, v/v) as an eluent to give target compounds.

General Procedure C for Preparation of Compound 63 (FIG. 1i)

To a solution of protected indole 1 (6.56 mmol) in 30 mL THF was added 2.0 M LDA solution (4.75 mL, 9.5 mmol) in THF within 10 min at −78° C., stirring at 0° C. for 30 min and subsequently cooled to −78° C. At this temperature, aryl aldehyde 60 (7.88 mmol), dissolved in dry THF (10 mL), was added. The resulting mixture was stirred overnight and allowed to warm to room temperature. The solution poured into 100 mL EtOAc. The combined organic layers were washed with sat. NH₄Cl, water and dried over anhydrous MgSO₄. The solvent was removed under reduced pressure and then purified by flash column chromatography on silica gel using EtOAc/Hexane (1/3, v/v) as an eluent to give compound 63.

General Procedure E for Preparation of Compounds 65 and 68 (FIGS. 1i and 1J)

To a solution of compound protected indole 64 and 67 (0.56 mmol) in 10 mL ethanol was added a 10% solution of NaOH (227 mg, 5.68 mmol) and the mixture was refluxed for 20 h. Then, ethanol was evaporated, brine and CH₂Cl₂ were added, and the organic phase extracted with CH₂Cl₂ and then purified by flash column chromatography on silica gel using EtOAc/Hexane (1/1, v/v) or CH₂Cl₂/Hexane (1/1, v/v) as an eluent to give target free indole compounds.

### Synthesis of 3′,4′,5′-Trimethoxy-biphenyl-3-carbaldehyde (Compound 60)

Method A (FIG. 1i);

Yield 91%;

MS (ESI) m/z 295.0 ([M+Na]⁺);

¹H NMR (CDCl₃) □ 10.10 (bs, 1H, CHO), 8.07 (t, J=1.7 Hz, 1H, ArH), 7.84 (m, 2H, ArH), 7.85 (t, J=7.8 Hz, 1H, ArH), 6.81 (s, 2H, ArH), 3.95 (s, 6H, OCH₃), 3.91 (s, 3H, OCH₃).

### Synthesis of (3′,4′,5′-Trimethoxy-biphenyl-3-yl)-(3,4,5-trimethoxy-phenyl)-methanol (Compound 61)

Method B (FIG. 1i);

Yield 71%;

MS (ESI) m/z 463.1 ([M+Na]⁺);

¹H NMR (CDCl₃) □ 7.60 (s, 1H, ArH), 7.47-7.31 (m, 3H, ArH), 6.76 (s, 2H, ArH), 6.64 (s, 2H, ArH), 5.81 (s, 1H, CH—OH), 4.00 (s, 6H, OCH₃), 3.90 (s, 3H, OCH₃), 3.81 (s, 9H, OCH₃), 2.97 (s, 1H, OH).

### Synthesis of (3′,4′,5′-Trimethoxy-biphenyl-3-yl)-(3,4,5-trimethoxy-phenyl)-methanone (Compound 62)

Method C (FIG. 1i);

Yield 85%;

MS (ESI) m/z 461.1 ([M+Na]⁺);

¹H NMR (300 MHz, CDCl₃) [symbol] 8.00 (t, J=1.5 Hz, 1H, ArH), 7.79 (m, 1H, ArH), 7.72 (dd, J=7.5, 1.5 Hz, 1H, ArH), 7.55 (t, J=7.5 Hz, 1H, ArH), 7.12 (s, 2H, ArH), 6.81 (s, 2H, ArH), 3.96 (s, 3H, OCH₃), 3.94 (s, 6H, OCH₃), 3.90 (s, 3H, OCH₃), 3.89 (s, 6H, OCH₃).

### Synthesis of (1-Benzenesulfonyl-1H-indol-2-yl)-(3′,4′,5′-trimethoxy-biphenyl-3-yl)-methanol (Compound 63)

Method D (FIG. 1i);

Yield 84%;

MS (ESI) m/z 552.2 ([M+Na]⁺);

¹H NMR (300 MHz, CDCl₃) [symbol] 8.00 (t, J=1.5 Hz, 1H, ArH), 7.79 (m, 1H, ArH), 7.72 (dd, J=7.5, 1.5 Hz, 1H, ArH), 7.55 (t, J=7.5 Hz, 1H, ArH), 7.12 (s, 2H, ArH), 6.81 (s, 2H, ArH), 3.96 (s, 3H, OCH₃), 3.94 (s, 6H, OCH₃), 3.90 (s, 3H, OCH₃), 3.89 (s, 6H, OCH₃).

### Synthesis of (1-Benzenesulfonyl-1H-indol-2-yl)-(3′,4′,5′-trimethoxy-biphenyl-3-yl)-methanone (Compound 64)

Method C (FIG. 1i);

Yield 85%;

MS (ESI) m/z 528.3 ([M+H]⁺);

¹H NMR (300 MHz, CDCl₃) [symbol] 8.21 (s, 1H, ArH), 8.17-8.06 (m, 3H, ArH), 7.94 (d, J=7.8 Hz, 1H, ArH), 7.82 (d, J=7.8 Hz, 1H, ArH), 7.58-7.46 (m, 6H, ArH), 7.34-7.32 (d, J=7.8 Hz, 1H, ArH), 7.01 (s, 1H, ArH), 6.83 (s, 2H, ArH), 3.95 (s, 6H, OCH₃), 3.91 (s, 3H, OCH₃).

### Synthesis of (1H-Indol-2-yl)-(3′,4′,5′-trimethoxy-biphenyl-3-yl)-methanone (Compound 65)

Method E (FIG. 1i);

Yield 75%;

MS (ESI) m/z 385.9 ([M−H]⁻);

¹H NMR (300 MHz, CDCl₃) [symbol] 9.62 (bs, 1H, NH), 8.17 (s, 1H, ArH), 7.98 (d, J=7.8 Hz, 1H, ArH), 7.82 (d, J=7.8 Hz, 1H, ArH), 7.73 (d, J=7.8 Hz, 1H, ArH), 7.61 (t, J=7.8 Hz, 1H, ArH), 7.52 (d, J=8.4 Hz, 1H, ArH), 7.40 (t, J=7.8 Hz, 1H, ArH), 7.21-7.16 (m, 2H, ArH), 6.86 (s, 2H, ArH), 3.95 (s, 6H, OCH₃), 3.93 (s, 3H, OCH₃).

### Synthesis of [3-(1-Benzenesulfonyl-1H-indol-2-yl)-phenyl]-(3,4,5-trimethoxy-phenyl)-methanol (Compound 66)

Method B (FIG. 1J);

Yield 71%;

MS (ESI) m/z 552.2 ([M+H]⁺);

¹H NMR (300 MHz, CDCl₃) [symbol] 8.30 (d, J=8.1 Hz, 1H, ArH), 7.63 (s, 1H, ArH), 7.49-7.16 (m, 12H, ArH), 6.71 (s, 2H, ArH), 6.56 (s, 1H, CH—OH), 5.87 (bs, 1H, CH—OH), 3.87 (s, 6H, OCH₃), 3.85 (s, 3H, OCH₃).

### Synthesis of [3-(1-Benzenesulfonyl-1H-indol-2-yl)-phenyl]-(3,4,5-trimethoxy-phenyl)-methanone (Compound 67)

Method C (FIG. 1J);

Yield 69%;

MS (ESI) m/z 550 ([M+Na]⁺);

¹H NMR (300 MHz, CDCl₃) [symbol] 8.31 (d, J=8.4 Hz, 1H, ArH), 8.02 (m, 2H, ArH), 7.73 (m, 1H, ArH), 7.64 (t, J=7.5 Hz, 1H, ArH), 7.50-7.31 (m, 8H, ArH), 7.27 (s, 2H, ArH), 6.65 (s, 1H, ArH), 4.01 (s, 3H, OCH₃), 3.99 (s, 6H, OCH₃).

### Synthesis of [3-(1H-Indol-2-yl)-phenyl]-(3,4,5-trimethoxy-phenyl)-methanone (Compound 68)

Method E (FIG. 1J);

Yield 95%;

MS (ESI) m/z 385.9 ([M−H]⁻);

¹H NMR (300 MHz, CDCl₃) [symbol] 8.94 (bs, 1H, NH), 8.17 (s, 1H, ArH), 7.94 (d, J=7.8 Hz, 1H, ArH), 7.69 (m, 2H, ArH), 7.55 (t, J=7.6 Hz, 1H, ArH), 7.41 (d, J=8.1 Hz, 1H, ArH), 7.25-7.15 (m, 2H, ArH), 7.12 (s, 2H, ArH), 6.92 (s, 1H, ArH), 3.98 (s, 3H, OCH₃), 3.85 (s, 6H, OCH₃).

### EXAMPLE 2

### Additional Synthetic Schemes for General Compound Analogs

Compound Analogs 14-20

Structural analogs of diindole 13 are shown in FIG. 1B. The structural analog compounds 14-20 are synthesized according to the general synthetic plan outlined in Schemes 2 through 4 (76-78) shown in FIGS. 1C-1E. For analog compound 14, a variety of substituted indole rings are prepared as shown in Scheme 2. To accomplish this, a variety of N-protected indoles 33 are synthesized from commercially available reagents and brominated at the 2-indole position to produce their corresponding bromides, 34. The bromides in turn are coupled via Suzuki reaction with aldehydoboric acid 4 to yield the corresponding aldehydo-indoles 35, key intermediates in this approach.

This class of aldehydo-indoles 5A, as shown in Scheme 3, are reacted with the 2-N-protected indole 1 under basic conditions to promote regioselective deprotonation and produce the hydroxymethylene compounds 8A in high yield. Corresponding methylketones 12A are then prepared by the oxidation of methanol linkage of compounds 8A with pyridinium dichromate (PDC) in DMF. De-protection of the N-protected groups affords a series of target indole products of basic structure 14 incorporating a variety of different substituents at varying positions in the indole system. For example, X may be halide, —OH, —OCH₃, CH₃, NO₂, CN, or CF₃.

For compounds 15 and 16 in FIG. 1B, aldehydo-indoles 38 linked at the 3-indole and 39 linked at the 4-, 5-, 6- or 7-indole position are prepared by respective Suzuki reactions of bromides 36 and 37 with aldehydoboric acid 4 as shown in Scheme 4 (FIG. 1D). The bi-phenyl 17, β-napthyl 18, substituted-aryl 19, and 3,4-methyelnedioxyphenyl 20 analogs shown in FIG. 1B are synthesized as shown in the bottom of Scheme 4, using procedures similar to those described above (5). These procedures provide a rapid, reliable and high yield synthetic method for the proposed compounds.

It is contemplated that other indole derivatives substituted at C3 in the indole ring may be synthesized. For example, indole may be derivatized with a substitutent at C3 that itself comprises a substituted thiazole ring. For example compound 59, methyl 2-(1H-indole-3-carbonyl)thiazole-4-carboxylate, would have the structure:

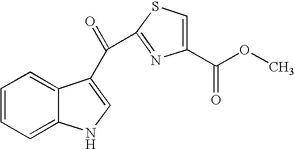

Analogs of compound 17, including compound 65, were synthesized as shown in FIG. 1i.

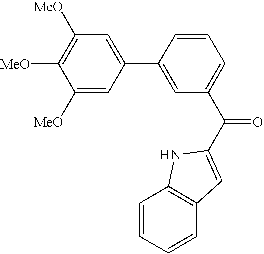

### Compound Analogs 21-23

Structural analogs 21-23, i.e., un-substituted and substituted derivatives of compounds 21, 22, and 23 (FIG. 1B) also are synthesized using the Suzuki reaction. However, in this case, the halogenated indoles are converted to lithium salts and then allowed to react with trimethyl borate to produce the needed boric acids 3, which are then reacted with the appropriate brominated aldehydo thiophene (X═S), furan (X═O), pyrrole (X═NH), or cyclopentadiene (X═CH₂) derivative 4B to yield a variety of heterocycle-linked diindoles 5B (Scheme 5; FIG. 1F). These derivatives are in turn be converted to diindoles with the corresponding hetercyclic linkages using lithium diisopropylamide (LDA) and PDC as shown in Scheme 3 (FIG. 1D). Linkages in the 2,4- and 2,5-positions will be synthesized in order to determine the importance of ring orientation and heterocyclic substitution on the benzyl linker position.

Analog 68, a trimethoxy derivative of 23, was synthesized as shown in FIG. 1J.

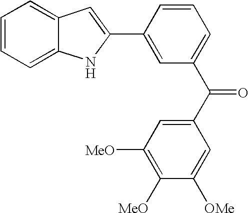

Compound Analogs 24-28

Analogs of compounds 24 through 28 (FIG. 1B) are synthesized to determine if the methylketone linkage is absolutely required for pharmacologic activity. A variety of thioketones 24, esters 25 and 27, and amides 26 and 28 are synthesized to explore the contributions of the hydrogen bond acceptor, length of the linkage, and position of the ketone, i.e., adjacent to the benzyl linker or indole ring) to activity. Thiophene analogs 24 are synthesized directly from their corresponding methylketone derivatives using hydrogen sulfide (Scheme 6; FIG. 1G) (6-7), while the ester and amide derivatives are made by reaction of the 2-amino 47 or 2-hydroxy-indoles 46 with 45 as previously described (8-10).

Compound Analogs 29-31

Analog compounds of 29-31, both substituted and unsubstituted derivatives (FIG. 1B) are synthesized to determine the structure-activity relationships for tubulin inhibition, anticancer activity, transport, and hepatic. Analogs are synthesized using reaction conditions as shown in Scheme 7 (FIG. 1H) which are nearly identical to those described in Schemes 2 through 6 (FIGS. 1C-1G), with the exception that the iodo-indole 56 will be lithiated and coupled with the brominated aldehydo compound 55 to give the corresponding alcohol, which is subsequently with PDC to the methylketone 57.

### EXAMPLE 3

In vitro and in vivo Methods

Cell viability (LNCaP, PC-3 prostate, DU145, PPC-1, and TSU-Pr1 prostate cancer cell lines, HT-29 colon cancer cell line, and MCF-7 breast cancer cell line) was quantitated using the sulforhodamine B (SRB) assay after 96 h coincubation with different concentrations of compound in 96-well plates. Cell viability of leukemia cells (K562 and doxorubicin-resistant K562/Dox) was quantitated by MTT assay after 96 h coincubation with different concentrations of compound in 96-well plates. Drug-induced apoptosis was determined by anti-histone ELISA assay and DNA laddering. Cell cycle progression was assessed by propidium iodide staining and fluorescence-activated cell sorting (FACS) analysis. In vitro tubulin polymerization assay was determined by CytoDYNAMIX ScreenTM3 (CDS-03) kits according to the manufacturer's instructions. Anti-apoptosis protein (Bcl-2 and Bcl-xl) and pro-apoptosis protein (Bax) were examined in LNCaP and PC-3 after 24 h incubation with different concentrations of compound 13 by Western blot assay. In vivo PC-3 xenograft studies were conducted by i.v. dosing of 50 mg/kg, 100 mg/kg and 150 mg/kg for 2 weeks.

### EXAMPLE 4

Effects of Various Compounds against Cancer Cell Lines in vitro

IC₅₀ of Different Cancer Cell Lines Treated with Compounds 13 and 68

Cells were plated in 96-well plates at a density of 800-5,000 cells/well, depending on the cell line, in their required growth media containing 10% fetal bovine serum. Preliminary studies were performed with each cell line using a variety of cell densities and incubation times to determine appropriate seeding densities. The compound of interest was dissolved in DMSO, diluted in cell culture medium (final DMSO concentration was less than 0.5% v/v), and added to quadruplicate wells at final concentrations ranging from 0 to 100 μM. Control wells to which only drug-free vehicle was added were included as negative controls.

Cells were incubated for 96 hour at 37° C. in a humidified atmosphere containing 5% carbon dioxide. Cell number at the end of drug treatment was quantified using the sulforhodamine B assay, as adopted by the National Cancer Institute (11) Cell survival at each drug concentration was calculated as the percentage of cells present as compared to that observed in vehicle-treated control wells, and the concentration that reduced cell number by 50% relative to the untreated control (i.e., the IC₅₀) was determined by nonlinear least squares regression using WinNonLin (Pharsight Corporation).

Different concentrations of precursor indole compounds 3 and 7 and novel diindole compounds 5, 8, 10, 11, 12, 13 were also tested. The IC₅₀ for known compounds indole and di(1H-indol-3-yl)methane and the 3-yl-boronic acid analog of compound 7 also were tested. Table 1 shows the IC₅₀ and Ki of tested compounds against various solid tumor cell lines, including four prostate cancer cell lines (LNCaP, PC-3, DU145, PPC-1), two bladder cancer cell line (TSU-Pr1 and TCCSUP), a colon cancer cell line (HT-29), a breast cancer cell (MCF-7), and a fibroblast cell line (CV-1).

Compound 13 has an IC₅₀ significantly lower than the control compound di(1H-indol-3-yl)methane or any other tested compound. Diindole 13 demonstrated potent growth inhibitory effects in all of the solid tumor cell lines tested, with IC₅₀ values ranging from 34 to 162 μM (Table 1). Diindoles 10 and 11 were significantly less potent in these cell lines. IC₅₀ values for diindole 10 ranged from 0.72 μM in HT-29 cells to >50 μM in the LNCaP, PC-3, and PPC-1 cell lines. Likewise, the IC₅₀ value for diindole 11 was 5.6 and 13.5 μM in the LNCaP and PC-3 cell lines, suggesting the importance of the methanone linkage, and possibly the presence of a hydrogen bond acceptor at this position, to anticancer activity. By comparison the IC50 values for paclitaxel in MCF-7 and HT-29 cells are about 2.5 nM (12). Compound 11, the indole derivative 3-(1H-indol-2-yl-)phenyl)methanol and an indole analog methyl 2-(1H-indole-3-carbonyl)thiazole-4-carboxylate were not yet tested (NT).

TABLE 1
In Vitro IC50 Values of Analogs in Various Human Cancer Cell Lines

IC₅₀ (μM)

| ID | Structure | LNCaP | PC-3 | DU-145 | PPC-1 | TSU | CV-1 |
|---|---|---|---|---|---|---|---|
| I-1 | 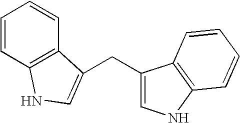 | 5.8 ± 0.5 | 39.4 ± 1.7 | 39.7 ± 2.6 | 31.8 ± 1.1 | 45.7 ± 1.1 | >100 |
| I-2 | 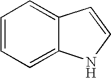 | >100 | >100 | >100 | >100 | >100 | ND |
| I-3 | 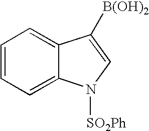 | 18.3 ± 1.4 | 59.9 ± 1.8 | 55.1 ± 2.5 | 41.1 ± 1.8 | 39.3 ± 1.6 | >100 |
| I-4 | 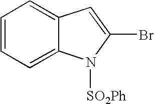 | 29.6 ± 2.2 | 69.3 ± 2.1 | 63.9 ± 1.9 | 52.1 ± 1.1 | 44.9 ± 2.0 | >100 |
| I-5 | 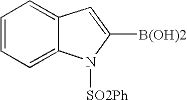 | 23.1 ± 4.0 | 73.9 ± 7.1 | 72.0 ± 3.4 | 80.8 ± 1.2 | 48.2 ± 3.4 | >100 |
| I-6 | 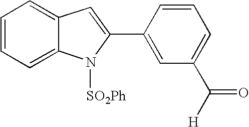 | 17.7 ± 1.2 | 23.2 ± 1.4 | 19.7 ± 1.3 | 13.9 ± 0.2 | 11.5 ± 0.1 | >100 |
| I-7 | 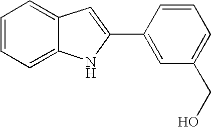 | 23.8 ± 3.0 | 65.1 ± 5.3 | ND | ND | ND | ND |
| I-8 | 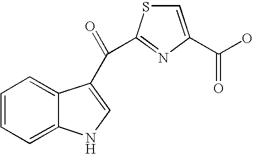 | 58.5 ± 14.3 | 90.4 ± 27.5 | ND | ND | ND | ND |
| I-9 | 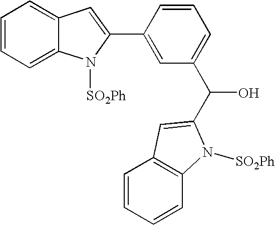 | >100 | >100 | >100 | 20-40 | >100 | >100 |
| I-10 | 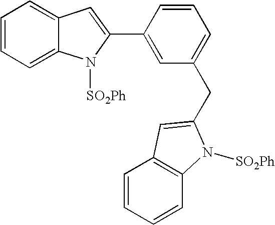 | >100 | >100 | >100 | >100 | >100 | ND |
| I-11 | 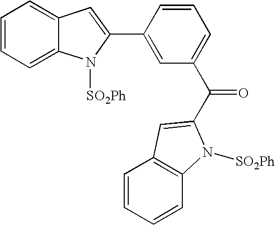 | >100 | >100 | >100 | >100 | >100 | >100 |
| I-12 | 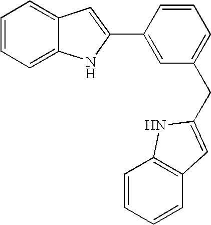 | >50 | >50 | N/A | >50 | 20-50 | >50 |
| 13 | 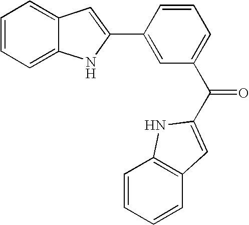 | .0442 ± .0024 | .0811 ± .0096 | .1381 ± .0126 | .0673 ± .0011 | .0338 ± .0018 | 0.0783 ± 0.035 |
| I-14 | 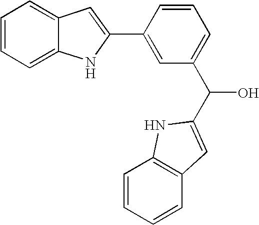 | 5.6 ± 1.1 | 13.5 ± 0.4 | ND | ND | ND | ND |
| I-15 | 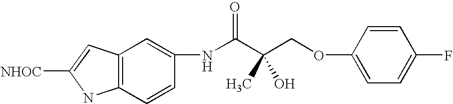 | >100 | >50 | N/A | >50 | 40.7 ± 2.8 | ND |
| 1-16 | 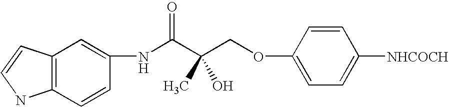 | >50 | >100 | N/A | >100 | >50 | ND |
| 68 | 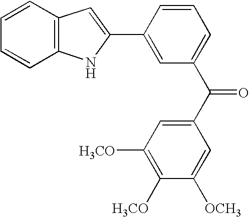 | 0.031 ± 0.0035 | 0.028 ± 0.002 | 0.023 ± 0.0041 | 0.019 ± 0.0015 | 0.018 ± 0.0021 | ND |

IC₅₀ of Different Drugs in K562 vs. K562/DOX Leukemia Cell Line

Cells were seeded in 96-well plates and incubated with different concentrations of compound 13, 68 or other anticancer drugs for 96 h. Cell viability was quantitated by MTT assay. The concentration that inhibited cell growth by 50% relative to the untreated control (IC50) was determined by nonlinear least squares regression using WinNonLin. Table 2 is a comparison of the IC50 of compound 12 and other anticancer drugs in the K562 and doxorubicin-resistant K562/DOX cell lines. The increase in IC50 for compounds 13 and 68 in the doxorubicin resistant cell line is minor compared to the increases for doxorubicin, vinblastine and taxol.

TABLE 2

| Compound | IC50 (nM) in K562 | IC50 (nM) in K562/DOX |
|---|---|---|
| Doxirubicin | 27.1 ± 5.5 | 859.3 ± 26.8 |
| Vinblastine | 0.3 ± 0.1 | 140.3 ± 10.1 |
| Taxol | 12.2 ± 0.2 | 1479.6 ± 479.8 |
| Cmpd 13 | 63.6 ± 2.4 | 78.2 ± 2.9 |
| Cmpd 68 | 23.6 ± 2.4 | 18.0 ± 4.4 |

Compound 13 Induced Apoptosis and DNA Fragmentation

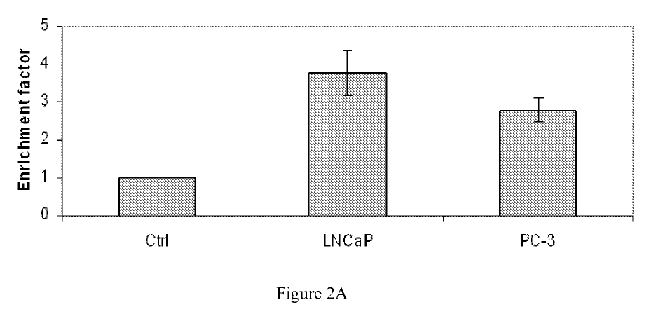

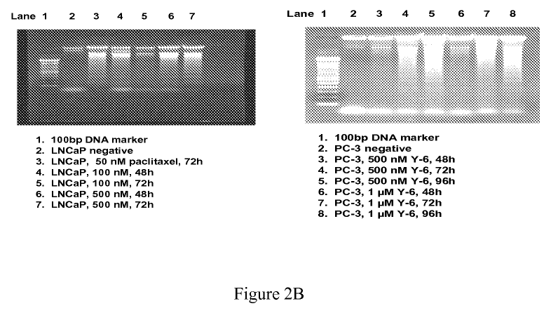

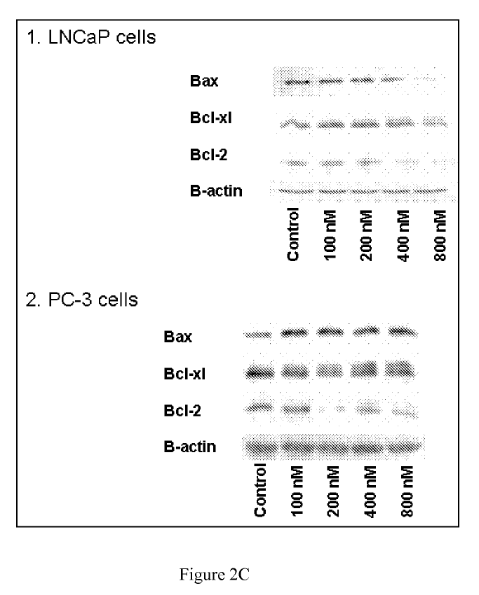

100 nM of compound 13 was incubated with LNCaP for 24 h and PC-3 for 48 h. Anti-histone ELISA detected apoptosis in the cell lines (FIG. 2A). The results are expressed as enrichment factor (Enrichment factor=OD of treated cells/OD from control cells). A Western blot of anti-apoptosis proteins, Bcl-2 and Bcl-xl, and pro-apoptosis protein Bax in LNCaP and PC-3 cells was performed. Bcl-2 was decreased by increasing concentration of compound 13 in both cell lines (FIG. 2B).

LNCaP and PC-3 cells were treated with different concentrations of drugs for different periods of time. At the end of the incubation, both floating and adherent cells were collected. Cells were lysed and low molecular weight DNA was precipitated and separated by 1.2% agarose gel electrophoresis. DNA was visualized by ethidium bromide staining and UV transillumination. Compound 13 induced DNA fragmentation in the cells (FIG. 2C).

Compound 13 Arrests LNCaP Cells in G2/M Phase and Inhibits Tubulin Polymerization

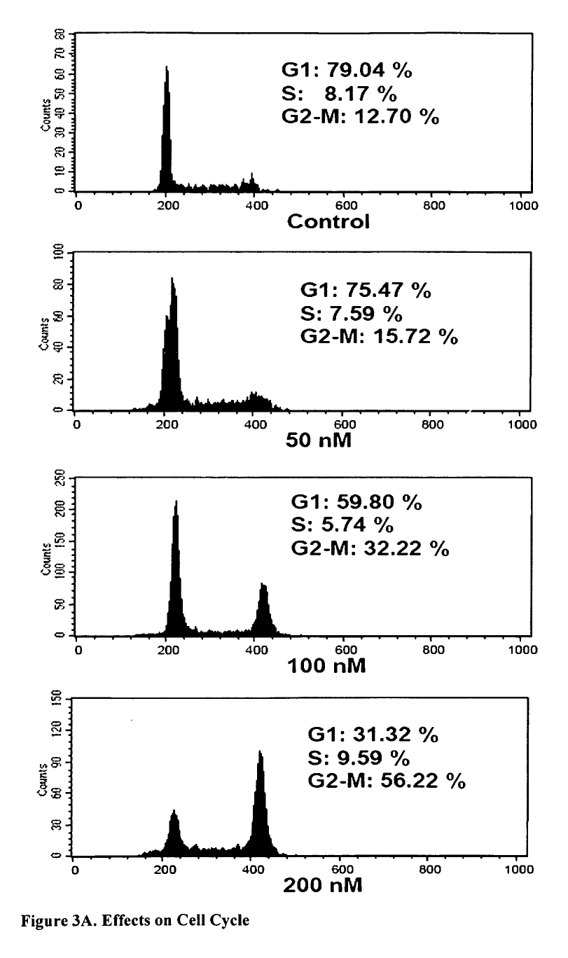

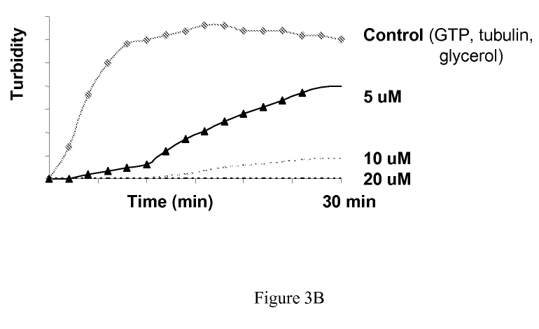

LNCaP cells were treated with 0, 50, 100 and 200 nM of compound 13 for 24 h (FIG. 3A). Cells were then harvested and fixed with 70% ethanol. Cell cycle distribution was determined by propidium iodide (PI) staining and analyzed by fluorescence-activated cell sorting (FACS) analysis.

Tubulin proteins (greater than 99% purity) were suspended (300 μg per sample) with 100 μl G-PEM buffer composed of 80 mM PIPES (piperazine-N,N′-bis(2-ethanesulfonic acid)), 2 mM MgCl₂, 0.5 mM egtazic acid and 1.0 mM guanosine triphosphate (GTP), pH 6.9, plus 5% glycerol in the absence or presence of the compound 12 at 4° C. The sample mixture was transferred to the prewarmed 96-well plate and absorbance was detected each minute for 30 minutes at 340 nm at 37° C. 20 μM of compound 13 can completely block the tubulin polymerization (FIG. 3B).

### EXAMPLE 5

Effects of Compound 13 against Cancer Cell Lines in vivo

Subchronic Toxicity Levels of Compound 13 in ICR Mice

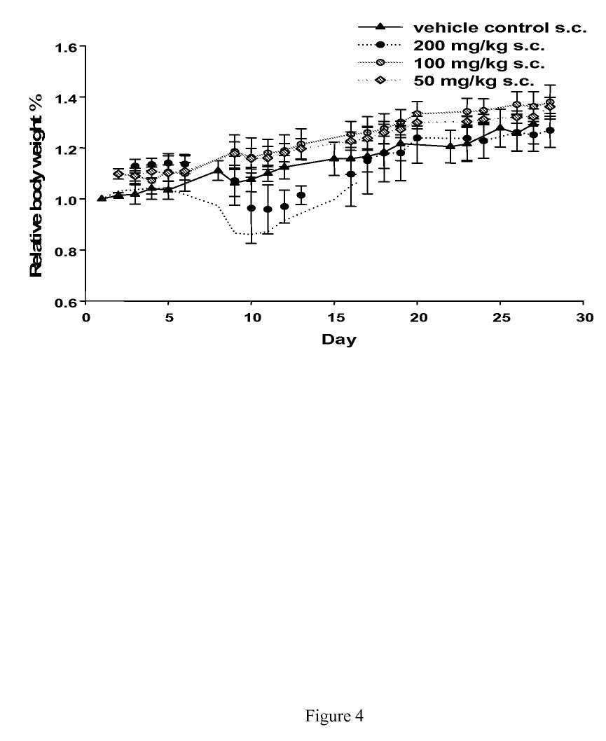

The maximally tolerated dose (MTD) in the mouse was identified. Doses of 50, 100, and 200 mg/kg (the limit of solubility in DMSO) were administered S.C. for 4 weeks (5-days on/2 days off), a commonly used regimen for initial preclinical studies of investigational anticancer agents (12). Body weight changes and morbidity in treated animals were used as a direct measure of toxicity. As shown in FIG. 4, all doses were generally well tolerated. There was no significant difference in morbidity or the rate of gain in body weight in animals treated with 50 or 100 mg/kg doses of diindole 13, while the highest dose caused 20% less body weight gain over the 4-week treatment period as compared to control animals treated with vehicle alone. These data suggested that diindole 13 was well tolerated, or that measurable plasma concentrations of the drug were not achieved due to rapid clearance.

Mean Plasma Concentration-Time Profile of Compound 13 in Mice

A single dose (10 mg/kg) and various routes of administration (intravenous, oral, and subcutaneous) were used in order to approximate its in vivo disposition and interpret the results of subchronic toxicity studies and forthcoming in vivo xenograft studies. Diindole 13 was administered to groups (n=60) of mice for each route of administration. Mice (n=5 per time point) were sacrificed at up to twelve different time points (pre-dose and up to 24 hours post-dose), and plasma samples were stored at −80° C. until HPLC analysis. An HPLC/UV analytical method was developed and validated to determine diindole 13 concentrations in plasma, with a linear range of 0.02 to 20 μg/mL and intra- and inter-day coefficients of variation at all concentrations less than 6%.

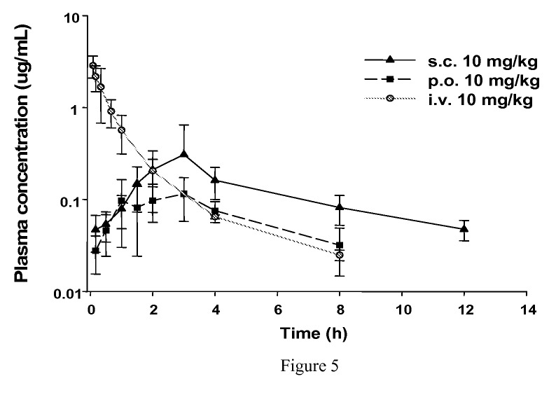

Plasma concentrations of bis-indole 13 declined rapidly after intravenous injection (FIG. 5), with a terminal half-life of less than 3 hours and clearance of about 4 L/h/kg (Table 3). Urine and fecal samples collected from mice after intravenous administration of diindole 13 showed that less than 5% of the drug was excreted unchanged in urine and feces. Plasma concentrations of diindole 13 peaked at about 3 hours after subcutaneous (S.C.) or oral (P.O.) administration, with absolute bioavailabilities of 73 and 29%, respectively. The terminal half-life after P.O. administration was similar to that observed after I.V. doses, but was longer after S.C. doses, likely reflecting slow absorption from the S.C. injection site due to limited aqueous solubility of diindole 13.

These data, coupled with estimates of the hepatic blood flow of the mouse (5.4 L/h/kg) (74), suggest that diindole 13 was extensively metabolized in the liver with a high hepatic extraction exceeding 0.75. Diindole 13 was widely distributed, with a volume of distribution about 10-fold larger than total body water (i.e., 0.6 L/kg). LC/MS/MS analysis of the metabolites in mice showed that diindole 13 undergoes extensive oxidative metabolism with subsequent sulfation (data not shown). Lastly, these data suggest that structural modifications, e.g., halogenation of the aromatic rings, to protect diindole 13 from microsomal oxidation via the hepatic cytochrome P450 may be beneficial. The pharmacokinetic parameters are provided in Table 3.

TABLE 3

| Parameters | I.V. | S.C. | P.O. |
|---|---|---|---|
| T½ (h) | 2.65 | 4.52 | 2.97 |
| AUC (mg * h/L) | 2.41 | 1.77 | 0.69 |
| Vss (L/Kg) | 6.35 | | |
| CL (L/h/Kg) | 4.1 | 5.7 | 14.3 |
| Tmax (h) | 0.08 | 3.0 | 3.0 |
| Cmax (mg/L) | 2.86 | 0.31 | 0.12 |
| F | 1.00 | 0.73 | 0.20 |

Antitumor Activity of Compound 13 in PC-3 Xenograft Balb/c Mice

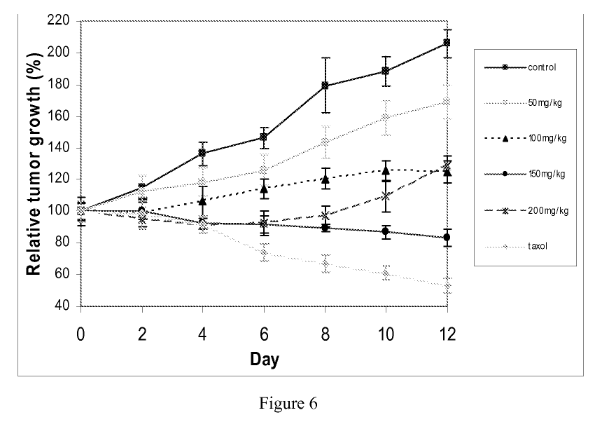

PC-3 tumor cells (2×10⁶ cells) were suspended in saline and injected S.C. in both flanks of recipient mice (n=15). Tumor size was measured every other day and volume calculated as V=π/6*(length)×(width)² (75). Daily treatment (5 days on/2 days off) was initiated with diindole 13 (50, 100, or 150 mg/kg/d) or paclitaxel (15 mg/kg/d for 4 days only due to toxicity as observed by decreased body weight) when tumors reached a volume of approximately 175 mm³. Tumor growth and body weight was monitored every other day for the remainder of the study. Paclitaxel (taxol) potently suppressed PC-3 xenograft growth at a dose of 15 mg/kg/d, but also elicited significant decreases in body weight (FIG. 6). Diindole 13 also suppressed tumor growth in a dose-dependent manner, with the 150 mg/kg/d dose approaching the antitumor efficacy and toxicity of paclitaxel.

### EXAMPLE 6

In vitro Chemosensitivity and Apoptosis Studies in Cells that Over-Express abc Transporters

These studies use pairs of parental and stably transfected or selection-maintained cell lines. For P-glycoprotein studies, the K562 leukemia (parental) and doxorubicin-resistant K562/Dox cell lines are used. For MRPx studies, the ovarian carcinoma 2008 cell line (parental) and its stably transfected variants that over-express MRP1 (2008 MRP1), MRP2 (2008 MRP2), and MRP3 (2008 MRP3) are used. These cells were provided by Professor Anton Berns of the Netherlands Cancer Institute. For BCRP studies, the HEK-293 (parental) and its stably transfected variant that over-expresses BCRP (ABCG2) are used and are obtained through Dr. Duxin Sun from Dr. Susan Bates, NIH. The chemosensitivity, i.e., IC₅₀ values, of each active compound is determined in these cell lines pairs as an initial assessment of the ability of these transporters to influence their activity.

Pilot experiments are conducted for each cell line using different seeding densities (1×10³ to 1×10⁶ cells per well) and incubation times to optimize growth conditions. Serial ten-fold dilutions (0.01 to 100 μM) are used. If necessary, smaller ranges of appropriate concentrations near the IC₅₀ for each drug are employed. Cell number in each well is determined using the SRB or MTT, for suspension cultures like K562 assay, and IC₅₀ values are determined using nonlinear regression (WinNonlin). The extent of transport is estimated as the ratio of IC₅₀ in ABC expressing cell line/IC₅₀ in parental cell line. Known substrates, e.g., calcein, mitoxantrone, and paclitaxel, and inhibitors, e.g., verapamil, sulfinpyrazone, and fumitremorgin C, are employed to assure the viability of the expressing cell lines and confirm the contribution of the specific transporter to resistance. Statistical comparisons of IC₅₀ values between compounds will be performed using ANOVA at a 5% level of significance.

Alternatively, drug transport in these cell lines can be conducted using HPLC or LC/MS/MS to quantify analog concentration, using methods similar to those previously reported to examine the structure-activity relationships for P-glycoprotein-mediated transport of steroidal glucocorticoids (13. In this instance, effective permeability coefficients and transport efficiency (T_{eff}) values are used for comparison.

### EXAMPLE 7

### Competition for Known Tubulin Binding Sites

A spin column binding assay, similar to that described by Bacher et al. (95-96) is used to determine whether diindoles compete for the same binding site as paclitaxel, colchicines, or vincristine. Depolymerized tubulin is incubated with radiolabeled paclitaxel, colchicine, or vincristine in the presence or absence of different concentrations (ranging from 0 to 20 μM) of unlabeled diindole 13 for 1 hour at 37° C. The incubate is then be loaded onto a size-exclusion Sephadex G25 column and centrifuged at 200×g for 1 min and the radioactivity in the flow-through will be quantified by scintillation counting. The column retains the free radioligand, but not the bound compounds. Thus, reduced radioactivity in the flow-through in the presence of diindole 13 indicates competitive binding. Unlabeled paclitaxel, colchicines, and vincristine are used as a positive controls.

Total radioactivity in each experiment is monitored for mass balance purposes. Heterocyclic or structurally modified analogs described herein that potently inhibit tubulin polymerization are used. If competition is observed, the equilibrium dissociation constant of each inhibitor (Kᵢ) for each agent is calculated by the following equation: Kᵢ=IC₅₀/(1+[L]/K_{d}), where IC₅₀ is the concentration of our ligand which inhibits the binding of ³H-radioligand by 50%, [L] is the concentration of ³H-radioligand added, and K_{d} is the equilibrium dissociation constant for the radioligand, e.g., ³H-vincristine. Experiments are performed in triplicate.

It is not expected that the binding of ³H-labeled paclitaxel, colchicine, or vincristine will be inhibited by diindole 13 or other compounds, based on the unique binding sites identified for other tubulin-interacting drugs (95-96). However, if they do, this provides another pharmacologic tool by which to examine the structure-activity relationships for tubulin interaction; namely, radioligand competition binding studies.

### EXAMPLE 8

In Vitro Hepatic Metabolism

For metabolite identification, diindole 13 and other compounds of interest are incubated with mouse liver S9 fraction (high protein concentration) with an NADPH-generating system, uridine diphosphoglucuronic acid (UDPGA) and other necessary cofactors at 37° C. for 2 h. A high protein concentration and long incubation time are chosen in order to assure maximal conversion of parent drug to metabolite(s), in the hope of identifying as many as possible, if not all, of the metabolites. Following incubation, proteins are precipitated with acetonitrile (v:v/1:1). The remaining organic phase in the supernatant is evaporated under nitrogen, and the resulting concentrated samples used for LC/MS/MS analysis.

Samples are analyzed using positive- and/or negative-ion electrospray ionization (ESI-) mass spectrometry (ThermoFinnigan LCQ DECA XP Max ion trap mass spectrometer, San Jose, Calif.). Gradient elution conditions for LC separation of the metabolites and optimized conditions for the mass spectrometer (e.g., capillary temperature, voltage, sheath and auxiliary gas flow, etc.) are determined in pilot experiments with each parent compound. Data acquisition is controlled by Xcalibur software (ThermoFinnigan) and metabolites are identified using Metabolite ID and Mass Frontier software. Synthetic standards are synthesized and independent NMR studies conducted where possible to confirm metabolite structure.

Preliminary studies using varying protein (i.e., microsome and S9) concentrations, drug concentration, and incubation time are performed to identify appropriate conditions for linear metabolite production and kinetic analyses. All reactions are conducted at 37° C. in the presence of NADPH and/or UDPGA (S9 fractions). The kinetic parameters, Km and Vmax, describing disappearance of the parent drug are determined by nonlinear regression analysis using WinNonlin (Pharsight) and the sigmoidal Emax model. Reactions are stopped by adding ice-cold acetonitrile (v:v/1:1) containing internal standard for HPLC or LC/MS/MS analysis. Protein present in the reaction mixture is precipitated by centrifugation and the supernatant either diluted with appropriate mobile phase or directly used for HPLC or LC/MS/MS analysis. HPLC and LC/MS/MS methods are developed and are validated for each analyte in each biological matrix and used for quantitation.

### EXAMPLE 9

### Acute and Subchronic Toxicity (Dose-Finding) Studies.

The maximally tolerated dose (MTD) and lethal dose to 10% of mice (LD₁₀) in male ICR mice (Taconic Laboratories) is determined. The analog of interest is dissolved in PEG300 or saline (as appropriate) at a concentration near its solubility, and serially diluted at 1:5 ratios to provide a range of dosing solutions. Animals receive progressively lower intravenous doses until the dose that does not result in the death or overt toxicity within 24 h is found, corresponding to the acute MTD (mg/kg). Less than 10 mice per drug are needed to establish the acute MTD.

To ensure that animal death during in vivo antitumor efficacy studies is due to tumor burden and not drug treatment, the subchronic toxicity of the analogs is determined. Mice are divided into groups of ten. Group 1 receives the acute MTD; group 2 receives 1/10 MTD; group 3: 1/25 MTD, group 4: 1/50 MTD; and group 5: 1/100 MTD. Doses are administered intravenously via the tail vein (to avoid concerns related to variable absorption after oral or subcutaneous injection) using a 5 days on/2 days off regimen for two consecutive weeks. The survival of mice is monitored for up to an additional 31 days following drug treatment. Plots of percent animals surviving versus dose (mg/kg) are constructed and the LD₁₀ determined by nonlinear regression. Studies with paclitaxel and vinblastine will also be performed.

### EXAMPLE 10

In Vivo Efficacy against K562 and K562/Dox Tumor Xenografts

K562 and K562/Dox tumor cells (generously provided by Dr. J. P. Marie, Paris, France) are mixed separately with Matrigel (Becton Dickinson) and injected subcutaneously (0.2 mL of cell and Matrigel suspension containing 1×10⁷ cells) into the left and right flank, respectively, of 8 week old male nude (nu/nu) mice. This allows one to simultaneously measure the response of both tumors to drug treatment in the same animal(s), reducing variability due to differences in body weight, pharmacokinetics, toxicity, etc. that may arise when comparing groups. Studies using tumor xenografts derived from cells that over-express other pertinent ABC transporters may also be included, if deemed pertinent.

Tumors are allowed to grow for approximately 3 weeks, with tumor volumes measured every other day, V=π/6*(length)×(width)² (75). Animals are randomized into treatment groups (n=10 per group) when tumor volumes reach 150 mm³. Ten animals per treatment group are required to assure adequate statistical power (0.8˜0.9) to identify a 25% difference in tumor volume between control and drug-treated groups. Five treatment groups are used for each compound: Group 1: un-treated control, Group 2: vehicle-treated control, Groups 3 to 5: treated with analog of interest at a daily intravenous dose of 0.01*LD₁₀, 0.1*LD₁₀, and the LD₁₀. Thus, the antitumor efficacy of each analog is examined in 50 nude mice bearing K562 and K562/Dox xenografts. Antitumor effect is assessed by measurement of tumor volume every other day during the experiments for up to 45 days after implantation, or when the tumor has reached a volume ≧10% of animal body weight. Tumor growth delay, rate of tumor growth, mean tumor volumes and final tumor volume will be compared between groups using ANOVA (α=0.05).

### EXAMPLE 11

Pharmacokinetics in Whole Animals

Male, ICR mice are used for these studies. Thirty animals receive an intravenous dose of the drug. Three mice are anesthetized and blood samples (about 500-1000 μL each) obtained via cardiac puncture or the orbital sinus at various times (up to 5 half-lives) after dosing. Plasma drug concentrations are determined using LC/MS methods (a ThermoFinnigan TSQ Quantum Discovery MAX triple quadrupole Mass Spectrometer and a LCQ Deca XP Max Ion Trap Mass Spectrometer are available in Dr. Dalton's lab, room 241). The area under the plasma drug concentration-time profile (AUC), volume of distribution, clearance and half-life is calculated for each group using nonlinear least squares regression and differences assessed using a two-tailed Student's t-test and multiple linear regression analysis. The pharmacokinetic advantage of diindole 13 and other analogs is assessed in tumor-bearing male nude nu/nu mice using a similar approach, with the exception that tumors are excised at these time points, and drug concentration in tumors containing the parental (K562) and P-glycoprotein expressing cells (K562/Dox) determined after homogenization and extraction. Maximal concentrations (Cmax) and AUCₜᵤₘₒᵣ values are compared using ANOVA.

The following references are cited herein.
1. Ketcha et al. J. Org. Chem. 1989, 54: 4350-4356.
2. Sakmoto et al. J. Chem. Soc., Perkin Trans. 1996, 1:1927-1934.
3. Mahboobi et al. J. Med. Chem. 2002, 45(5):1002-1018.
4. Mahboobi et al. J. Org. Chem. 1999, 64:8130-8137.
5. Hashizume et al. Chem Pharm Bull (Tokyo), 1994, 42(10):2097-2107.
6. Elofson et al. J. Org. Chem. 1964, 29.
7. Paquer, D. and Vialle, J. Bulletin de la Societe Chimique de France, 1969, 10:3595-3601.
8. Li et al. Chemistry, 2000, 6(9):1531-1536.
9. Maugard et al. Phytochemistry, 2001, 58(6):897-904.
10. Venepalli et al. J Med Chem, 1992, 32(2):374-378.
11. Rubinstein et al. J Natl Cancer Inst, 1990, 82(13):1113-1118.
12. Rose, W. C. Taxol: Science and Applications, M. Suffness, Editor. 1995, CRC Press: Boca Raton, Fla. p. 209-235.
13. Yates et al. Pharm Res, 2003, 20(11):1794-1803.

It will be obvious to those skilled in the art that various changes may be made without departing from the scope of the invention, which is not to be considered limited to what is described in the specification.

What is claimed is:

1. A compound having the structural formula: 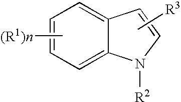
where:
R¹ is H, halide, CF₃, NO₂, OH, —OCH₃, or CN alkyl, alkenyl, O-alkyl, and O-aryl, and n is 0, 1, 2, 3, or 4;
R² is H or —SO₂Ph;
R³ is phenyl substituted at C3 or C5 with R⁴; R⁸R⁹; R¹²R¹³; 2-, 3- or 6-indolyl substituted at C1, C2, or C3 with 2-, 3- or 6-indolyl, either of said indolyl moiety independently substituted at C1 with R², at C4, C5, or C6 with R¹ or with a combination thereof; or naphthyl substituted at C5, C6, or C7 with 2-, 3- or 6-indolyl or unsubstituted, said indolyl moiety independently substituted at C1 with R², at C4, C5, or C6 with R¹ or with a combination thereof;
R⁴ is R⁵; C₁₋₃alkylene-R⁵; C(O)R⁶; CH═CH—C(R⁷)—R⁶; —C(O)—R⁷—R⁶; —O—C(R⁷)—R⁶; R^{8;} R⁷R⁸-(2-, 3-, or 6-indolyl); R⁸-(2-, 3- or 6-indolyl), said indolyl moiety independently substituted at C1 with R², at C4, C5 or C6 with R¹ or with a combination thereof; R⁸R⁹or R¹²R¹³;
R⁵ is OH, NO₂, NH₂, —NH—C₁₋₃alkyl, N═N═N, CN, or OR⁶;
R⁶ is H, C₁₋₃alkyl, or a 5- or 6-membered ring independently substituted at C2, C3, C4, C5, or C6 with R1;
R⁷ is O, S or NH;
R⁸ is —CH₂, —CH₂OH, C═O, C═S, C═CH₂, C═NOH, C═N(NH₂);
R⁹ is phenyl independently substituted at C3 with R¹⁰ and at C4 and C5 with R¹¹; thiazolyl substituted at C4 with —C(O)OCH₃ or naphthyl substituted at C5, C6, or C7 with 2-, 3- or 6-indolyl or unsubstituted, said indolyl moiety independently substituted at C1 with R², at C4, C5, or C6 with R¹ or with a combination thereof;
R¹⁰ is H, OH, —OCH₃, phenyl, naphthyl or forms a dioxolyl ring with R¹¹ at C4;
R¹¹ is H, OH, or —OCH₃;
R¹² is pyrrolyl, furanyl, thienyl, or cyclopentadienyl;
R¹³ is —C(O)-2-, 3-, or 6-indolyl, —C(O)-imidazole, —C(O)-thiazole, —C(O)-oxazole, —C(O)-isoxazole, —C(O)-benzoxazole, —C(O)-pyrrole, —C(O)-furan, —C(O)-oxazoline, —C(O)-oxazolidine, —C(O)-oxadiazole, C(O)-napthyl or —C(O)phenyl, each independently substituted with at C2, C3, C4, C5, or C6 with R¹; and
a pharmacologically acceptable salt or hydrate thereof.

2. The compound as claimed in claim 1, wherein R¹ is H, R³ is phenyl substituted at C3 or C5 with R⁴, and R⁴ is with R⁸.

3. The compound as claimed in claim 2 having a structure selected from:

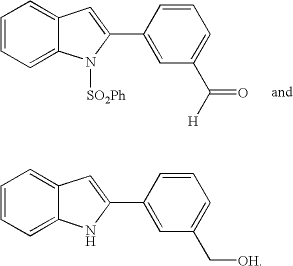

4. The compound as claimed in claim 1, wherein R¹ is H or F, R³ is phenyl substituted at C3 or C5 with R⁴, R⁴ is R⁸-(2-, 3- or 6-indolyl).

5. The compound as claimed in claim 4 having a structure selected from:

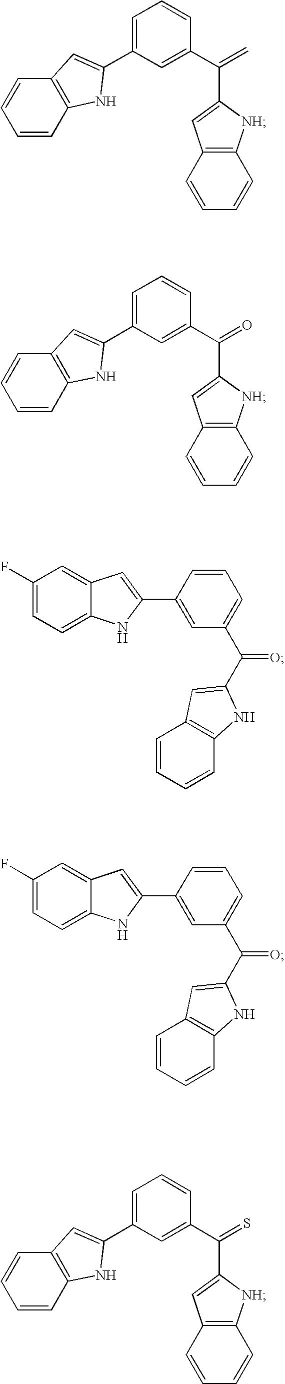

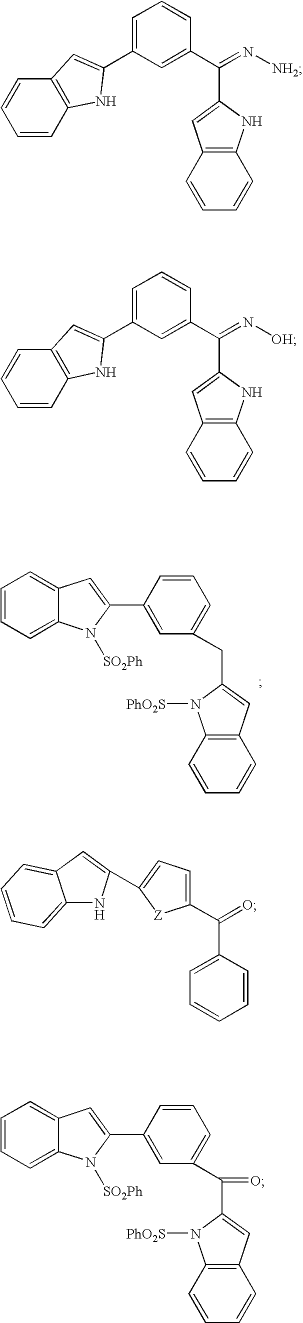

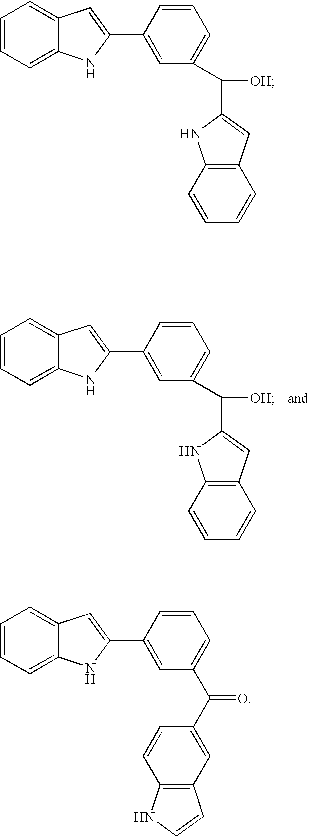

6. The compound as claimed in claim 1, wherein R³ is phenyl substituted at C3 or C5 with R⁴ and R⁴ is R⁷R⁸-(2-, 3-, or 6-indolyl).

7. The compound as claimed in claim 6 having a structure selected from:

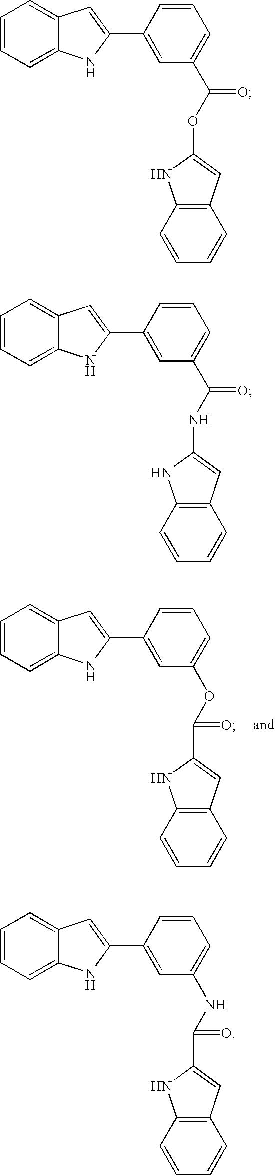

8. The compound as claimed in claim 1, wherein R³ is phenyl substituted at C3 or C5 with R⁴ and R⁴ is R⁸R⁹.

9. The compound as claimed in claim 8 having a structure selected from:

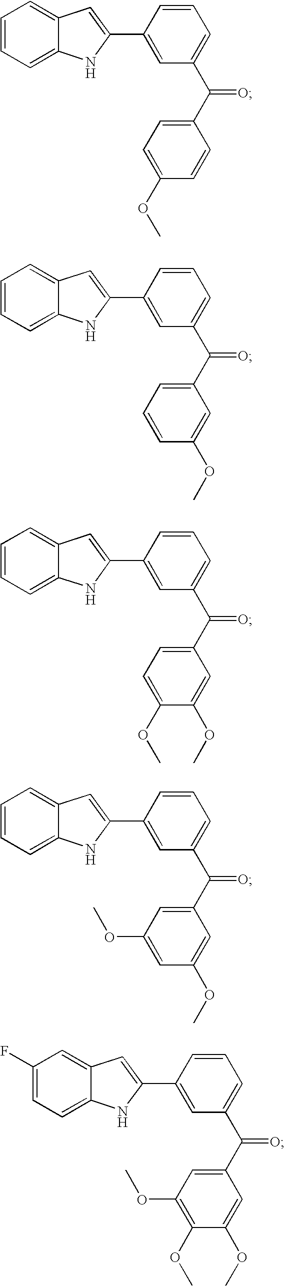

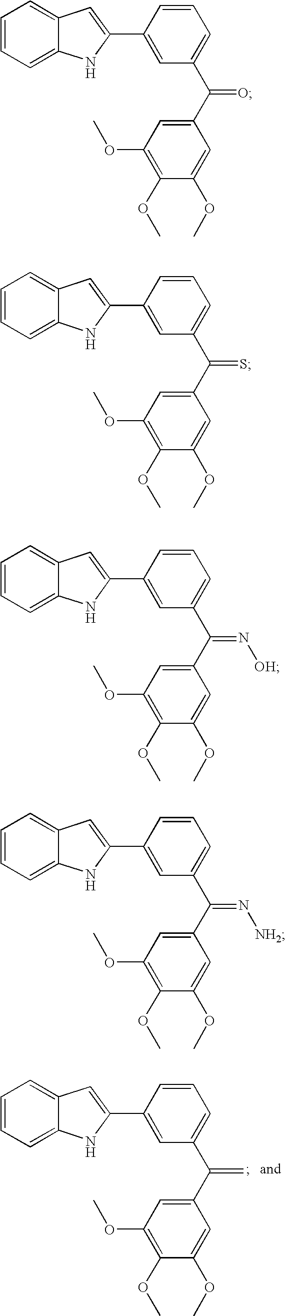

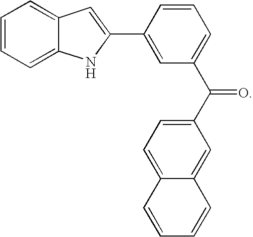

10. The compound as claimed in claim 8 having a structure of

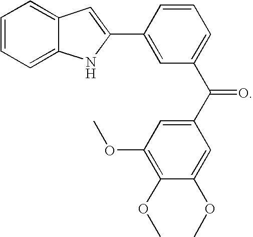

11. The compound as claimed in claim 1 wherein R³ is 2-, 3- or 6-indolyl.

12. The compound as claimed in claim 11 having a structure selected from:

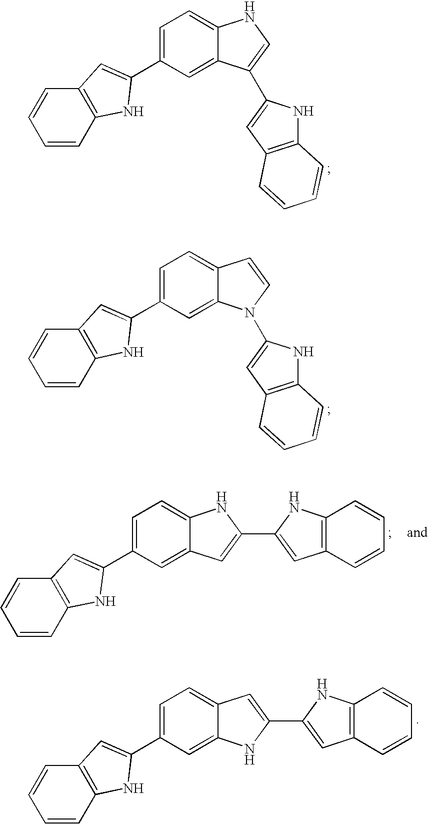

13. The compound as claimed in claim 1, wherein R³ is napthyl.

14. The compound as claimed in claim 13 having a structure selected from:

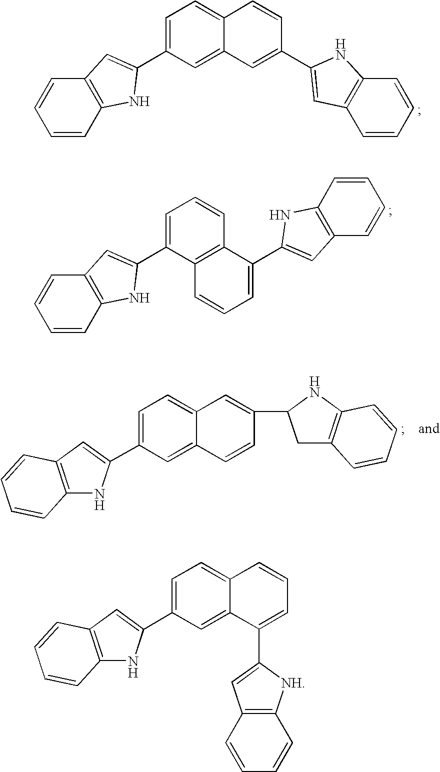

15. The compound as claimed in claim 1, wherein R³ is R⁸R⁹.

16. The compound as claimed in claim 15 having a structure selected from: 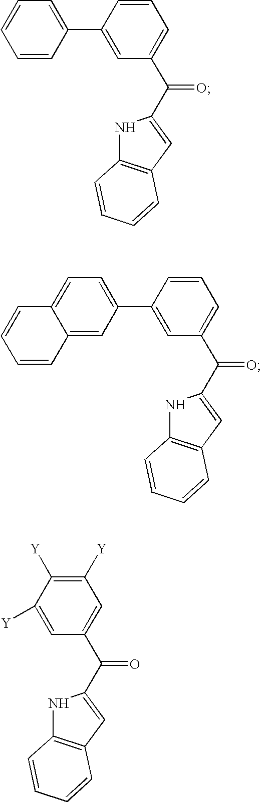
wherein Y is independently selected from H, OH, OCH₃; and

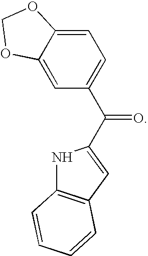

17. The compound as claimed in claim 1, wherein R³ is R¹²R¹³.

18. The compound as claimed in claim 17 having a structure selected from: 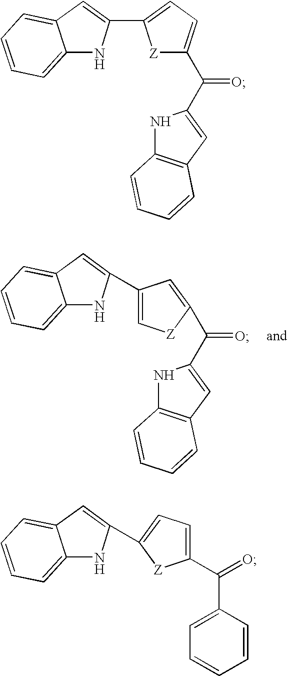
and wherein Z is independently selected from S, O, NH, and CH₂.

19. A method of inhibiting tubulin polymerization in a cell associated with a cell proliferative disease, comprising:
contacting said cell with a pharmacologically effective amount of a compound of claim 1.

20. The method of claim 19, wherein said cell proliferative disease is a cancer.

21. The method of claim 20, wherein said cancer is prostate cancer, colon cancer or breast cancer.
